(19) United States
(12) Patent Application Publication
(10) Pub. No.: US 2023/0047927 A1
(43) Pub. Date: Feb. 16, 2023

(54) SYSTEMS AND METHODS FOR FLOW CELL SAMPLE ALLOCATION

(71) Applicant: Tempus Labs, Inc., Chicago, IL (US)

(72) Inventors: Adrian Garcia Badaracco, Chicago, IL (US); Mitchell Berrie, Chicago, IL (US)

(21) Appl. No.: 17/400,117

(22) Filed: Aug. 12, 2021

(51) Int. Cl. G01N 35/00

(52) U.S. Cl. CPC G01N 35/0095; G01N 35/00732; G01N 2035/0094; G01N 2035/00752

(57) ABSTRACT

A method and system for pooling a plurality of specimens for processing, each specimen associated with a set of specimen characteristics. Each specimen is grouped based on the set of specimen characteristics, where the set of specimen characteristics includes a mass of each specimen. A set of flow cell characteristics for each flow cell included in a group of flow cells that includes at least one flow cell is identified. At least one pool is generated based on the set of specimen characteristics associated with each specimen included in the plurality of specimens and the set of flow cell characteristics for each flow cell included in the group of flow cells. Each pool is associated with a lane included in a flow cell and includes at least one specimen included in the plurality of specimens, and each lane is associated with a specimen type.

### CROSS-REFERENCE TO RELATED APPLICATIONS

N/A.

### BACKGROUND

Flow cells can be used to sequence genetic material such as DNA and RNA. A flow cell can include a number of lanes that samples of genetic material can be allocated into. The genetic material can be a sample taken from a tumor. Samples from multiple patients and/or biological regions can be positioned in the lanes of the flow cell and then sequenced.

Previously, flow cell allocation has been a manual process where humans (e.g., lab technicians) arrange samples according to their best guess of what samples should be run together in the same flow cell. Unfortunately, manual allocation can be tedious, slow, and/or subject to human error.

In particular, the process of flow cell allocation for sequencing includes selecting samples to be sequenced, selecting flowcells to be used in the sequencing process, and arranging the samples in the flowcells once both the samples and the flowcells have been selected. One type of sequencing may be next-generation sequencing, which produces millions of short reads (e.g., sequence reads) or long reads for each biological sample. Accordingly, in some embodiments, the plurality of sequence reads obtained by next-generation sequencing of cfDNA molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides. Each of these steps can be affected by a plurality of options. For example, flow cells generally include a number of constraints which can dictate how samples are allocated within the flow cell. When each of these steps is coupled with the factors affecting each step, it will be seen that the number of possible allocation options increases exponentially, with those options numbering in the trillions. Thus, it will be appreciated that brute force methods to address those constraints may be prohibitive to the process of allocating samples in a timely manner, in addition to being costly in terms of computational resources.

Accordingly, methods of positioning samples within one or more flow cells that address one or more of these issues are needed.

### SUMMARY OF DISCLOSURE

Disclosed herein are systems, methods, and mechanisms useful for automatically determining how to position samples in one or more flow cells. In particular, the disclosure provides systems, methods, and mechanisms for allocating samples to specific locations in one or more flow cells based on sample characteristics, flow cell characteristics, and one or more constraints.

In accordance with some embodiments of the disclosed subject matter, a method of pooling a plurality of specimens for processing, each specimen included in the plurality of specimens associated with a set of specimen characteristics is provided. The method includes grouping each specimen in the plurality of specimens based on the set of specimen characteristics associated with each specimen included in the plurality of specimens, identifying a set of flow cell characteristics for each flow cell included in a group of flow cells comprising at least one flow cell, and generating at least one pool based on the set of specimen characteristics associated with each specimen included in the plurality of specimens and the set of flow cell characteristics for each flow cell included in the group of flow cells, wherein each pool is associated with a lane included in a flow cell included in a group of flow cells and comprises at least one specimen included in the plurality of specimens, and each lane included in the group of flow cells is associated with a specimen type.

In accordance with some embodiments of the disclosed subject matter, a specimen pooling system comprising at least one processor and at least one memory is provided. The memory comprises instructions to group each specimen in a plurality of specimens based on a set of specimen characteristics associated with each specimen included in the plurality of specimens, identify a set of flow cell characteristics for each flow cell included in a group of flow cells comprising at least one flow cell, and generate at least one pool based on the set of specimen characteristics associated with each specimen included in the plurality of specimens and the set of flow cell characteristics for each flow cell included in the group of flow cells, wherein each pool is associated with a lane included in a flow cell included in a group of flow cells and comprises at least one specimen included in the plurality of specimens, and each lane included in the group of flow cells is associated with a specimen type.

### BRIEF DESCRIPTION OF DRAWINGS

FIG. 1 is an example of a system for allocating samples.

FIG. 2 is an example of hardware that can be used in some embodiments of the system for allocating samples in FIG. 1.

FIG. 3 is an exemplary embodiment of a flow for sequencing samples in one or more flow cells.

FIG. 4 is an exemplary search tree that can be used to search for suitable groups of one or more flow cells for allocating a plurality of samples.

FIG. 5 is an example of flow cell sample configurations.

FIG. 6 is an example of a first flow cell sample configuration and a second flow cell sample configuration each having multiple pools.

FIG. 7 is an exemplary flow for generating a plurality of pools based on a plurality of samples.

FIG. 8 an exemplary process that can allocate samples in a flow cell based on sample data and flow cell data.

FIG. 9 is an exemplary search tree that has been sorted by processing cost.

FIG. 10 is another exemplary search tree.

FIG. 11 is an exemplary process 1100 for implementing a specimen mapping system and/or a sequencing system

### DETAILED DESCRIPTION

The various aspects of the subject disclosure are now described with reference to the drawings. It should be understood, however, that the drawings and detailed description hereafter relating thereto are not intended to limit the claimed subject matter to the particular form disclosed. Rather, the intention is to cover all modifications, equivalents, and alternatives falling within the spirit and scope of the claimed subject matter.

In the following detailed description, reference is made to the accompanying drawings which form a part hereof, and in which is shown by way of illustration, specific embodiments in which the disclosure may be practiced. These embodiments are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions, rearrangements, or combinations thereof within the scope of the disclosure may be made and will become apparent to those of ordinary skill in the art.

In accordance with common practice, the various features illustrated in the drawings may not be drawn to scale. The illustrations presented herein are not meant to be actual views of any particular method, device, or system, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or method. In addition, like reference numerals may be used to denote like features throughout the specification and figures.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof. Some drawings may illustrate signals as a single signal for clarity of presentation and description. It will be understood by a person of ordinary skill in the art that the signal may represent a bus of signals, wherein the bus may have a variety of bit widths and the disclosure may be implemented on any number of data signals including a single data signal.

The various illustrative logical blocks, modules, circuits, and algorithm acts described in connection with embodiments disclosed herein may be implemented as electronic hardware, computer software, or combinations of both. To clearly illustrate this interchangeability of hardware and software, various illustrative components, blocks, modules, circuits, and acts are described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the embodiments of the disclosure described herein.

In addition, it is noted that the embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a function, a procedure, a subroutine, a subprogram, etc. Furthermore, the methods disclosed herein may be implemented in hardware, software, or both. If implemented in software, the functions may be stored or transmitted as one or more instructions or code on a computer-readable medium. Computer-readable media includes both computer storage media and communication media including any medium that facilitates transfer of a computer program from one place to another.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, the terms “component,” “system” and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a computer and the computer can be a component. One or more components may reside within a process and/or thread of execution and a component may be localized on one computer and/or distributed between two or more computers or processors.

The word “exemplary” is used herein to mean serving as an example, instance, or illustration. Any aspect or design described herein as “exemplary” is not necessarily to be construed as preferred or advantageous over other aspects or designs.

Furthermore, the disclosed subject matter may be implemented as a system, method, apparatus, or article of manufacture using standard programming and/or engineering techniques to produce software, firmware, hardware, or any combination thereof to control a computer or processor-based device to implement aspects detailed herein. The term “article of manufacture” (or alternatively, “computer program product”) as used herein is intended to encompass a computer program accessible from any computer-readable device, carrier, or media. For example, computer readable media can include but are not limited to magnetic storage devices (e.g., hard disk, floppy disk, magnetic strips, etc.), optical disks (e.g., compact disk (CD), digital versatile disk (DVD), etc.), smart cards, and flash memory devices (e.g., card, stick).

Additionally, it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope or spirit of the claimed subject matter.

As used herein, “cancer” shall be taken to mean any one or more of a wide range of benign or malignant tumors, including those that are capable of invasive growth and metastases through a human or animal body or a part thereof, such as, for example, via the lymphatic system and/or the blood stream. As used herein, the term “tumor” includes both benign and malignant tumors and solid growths. Typical cancers include but are not limited to carcinomas, lymphomas, or sarcomas, such as, for example, ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans.

The present disclosure provides a method for automatically allocating flow cells by eliminating infeasible choices in an efficient manner, as well as efficiently determining least costly (e.g., according to one or more constraints) feasible solutions. As noted above, brute force methods to address those constraints can require trillions of iterations, so that efficient allocation of samples in a timely manner is not achieved by merely implementing those methods using a computer, since doing so without more would still be unacceptably costly in terms of computational resources. Instead, as described herein, the present disclosure presents systems and methods that address those drawbacks to provide an efficient, practically applicable allocation of flow cells.

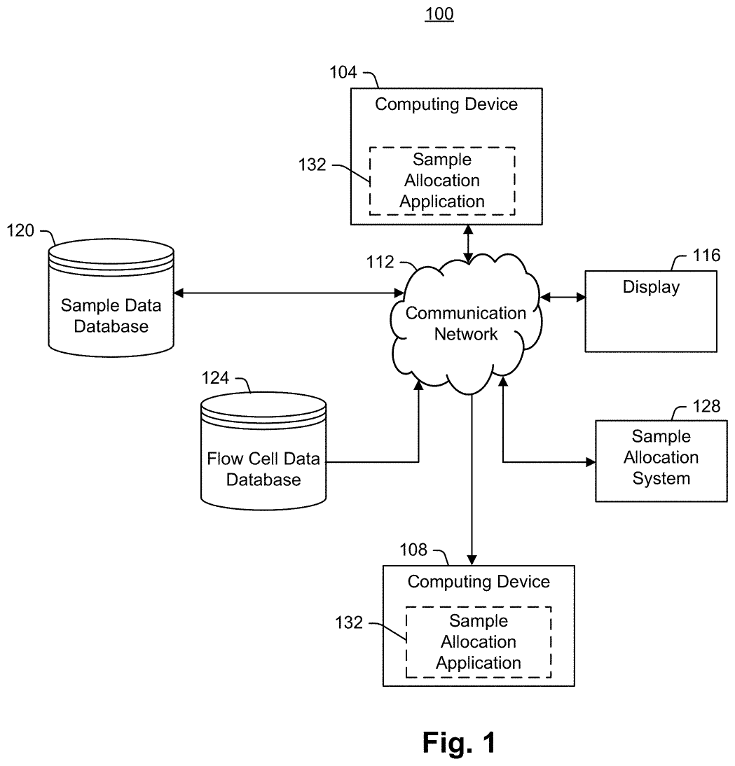

With reference now to the figures, FIG. 1 shows an example of a system 100 for allocating samples to a multiplicity of flow cells. In some embodiments, the system 100 can include a computing device 104, a secondary computing device 108, and/or a display 116. In some embodiments, the system 100 can include a sample data database 120, a flow cell data database 124, and/or a trained models database 128. In some embodiments, the trained models database 128 can include one or more trained machine learning models such as artificial neural networks. In some embodiments, the computing device 104 can be in communication with the secondary computing device 108, the display 116, the sample data database 120, the flow cell data database 124, and/or the trained models database 128 over a communication network 112. As shown in FIG. 1, the computing device 104 can receive sample data and/or flow cell data.

The sample allocation application 132 can be included in the secondary computing device 108 that can be included in the system 100 and/or on the computing device 104. The computing device 104 can be in communication with the secondary computing device 108. The computing device 104 and/or the secondary computing device 108 may also be in communication with a display 116 that can be included in the system 100 over the communication network 112.

The communication network 112 can facilitate communication between the computing device 104 and the secondary computing device 108. In some embodiments, communication network 112 can be any suitable communication network or combination of communication networks. For example, communication network 112 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, etc.), a wired network, etc. In some embodiments, communication network 112 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

The sample data database 120 can include sample data associated with a plurality of specimens. In some embodiments, the sample data can include, for each sample included in the plurality of specimens, a set of sample characteristics including a mass, a unique identifier such as a barcode, a source tissue type, an age, a turn-around-time, and/or a sample type.

The flow cell database 124 may include data pertaining to one or more of the various flow cells that are available for sequencing. For example, that data can include a quantification of flow cell channels/lanes in the flow cell, density of flow cell wells in each channel, lower and/or upper bounds for supported read lengths per channel/lane, minimum and/or maximum number of mass for each specimen to be sequenced, minimum number of flow cell wells which must be occupied in each channel, supported systems of sequencer, number of controls and libraries, supported run-times per flow cell, and other flow cell characteristics.

In one example, a sequencer may support four different flow cells, such as the four types of flow cells available for sequencing on the NovaSeq platform: the S4 flow cell (four lanes per flow cell), the S2 flow cell (two lanes per flow cell), the S1 flow cell (two lanes per flow cell), and the SP flow cell (two lanes per flow cell). Each having a supported number of read-pairs per lane, including: the S4 flow cell to deliver 2.0 to 2.5 billion read-pairs per lane; the S2 flow cell to deliver 1.6 to 2.0 billion read-pairs per lane; the S1 flow cell to deliver 600-800 million read-pairs per lane; and the SP flow cell to deliver 300-400 million read-pairs per lane. Flow cells may support any number of sequence read lengths, such as sequence runs with read lengths of either 50×50 bp, 100×100 bp, 150×150 bp, or 250×250 bp. In one example, the system supports read lengths up to 250×250 bp on an SP flow cell and the other classes of flow cells (S1, S2, and S4) support read lengths up to 150×150 bp. Flow cells may include library loading molarity requirements such as a volume of 18 μl (SP flow cell lane) to 30 μl (S4 flow cell lane) of a 1.0 to 1.5 nM library including whether the flow cell has support for additional volume to perform quality control analysis (Qubit assay, Agilent TapeStation assay and qPCR).

Laboratories performing next-generation sequencing may reference the characteristics of the flow cell to identify specimen quantity and/or mass lower bounds and upper bounds for each flow cell based upon their assay performance and tolerance for risk of failure or reduced quality in the sequencing results. In one example, where a lane may be used for tumor or normal specimen, normal lanes may be determined to have to be at least 35% full and tumor lanes at least 80% full to generate high-quality sequencing results. In this example, depending on the configuration of the flow cell, you'd have to multiply the minimum quantity of specimen tissue by the number of lanes assigned to that flow cell type. In exemplary lane distributions, an SP flow cell with tumor specimen in both lanes will have a capacity 2× the lane minimum provided, an S4 flow cell with one normal lane and three tumor lanes will have a capacity 1× the normal minimum for an S4 and 3× the minimum tumor lanes provided. Where the minimums per lane may be configured to:

SP lane: a minimum of 17 normal specimen per lane and a minimum of 12 tumor specimen per lane;

S1 lane: a minimum of 33 normal specimen per lane and a minimum of 25 tumor specimen per lane;

S2 lane: a minimum of 84 normal specimen per lane and a minimum of 64 tumor specimen per lane; and

S4 lane: a minimum of 102 normal specimen per lane and a minimum of 39 tumor specimen per lane.

While the numbers presented herein include the examples above, other minimums may be assigned keeping the tumor and normal quantities the same, or having new lower bounds based on improvements to the underlying sequencing devices that enable high-quality results or based upon a laboratory's tolerance for lower quality results or sequencing fails.

Exemplary flow cell characteristics for one sequencer are summarized in Table 1:

TABLE 1

| Flow | | | | |
|---|---|---|---|---|
| cell type | SP | S1 | S2 | S4 |
| Lanes per | 2 | 2 | 2 | 4 |
| flow cell | | | | |

Output per flow cell

| | | | | |
|---|---|---|---|---|
| 1 × 35 bp | N/A | N/A | N/A | 280-350 Gb |
| 2 × 50 bp | 65-80 Gb | 134-167 Gb | 333-417 Gb | N/A |
| 2 × 100 bp | 134-167 Gb | 266-333 Gb | 667-833 Gb | 1600-2000 Gb |
| 2 × 150 bp | 200-250 Gb | 400-500 Gb | 1000-1250 Gb | 2400-3000 Gb |
| 2 × 250 bp | 325-400 Gb | N/A | N/A | N/A |
| Single-end | 650-800M | 1.3-1.6 B | 3.3 B-4.1 B | 8-10 B |
| reads clusters | | | | |
| passing filter | | | | |
| (CPF) | | | | |
| Paired-end | 1.3-1.6 8 | 2.6-3.2 B | 6.6-8.2 B | 16-20 B |
| reads CPF | | | | |

Quality scores

| | |
|---|---|
| 1 × 35 bp | ≥90% of bases higher than Q30 at 1 × 35 bp |
| 2 × 50 bp | ≥90% of bases higher than Q30 at 2 × 50 bp |
| 2 × 100 bp | ≥85% of bases higher than Q30 at 2 × 100 bp |
| 2 × 150 bp | ≥85% of bases higher than Q30 at 2 × 150 bp |
| 2 × 250 bp | ≥75% of bases higher than Q30 at 2 × 250 bp |

Run time

| | | | | |
|---|---|---|---|---|
| 1 × 35 bp | N/A | N/A | N/A | ~14 hr |
| 2 × 50 bp | ~13 hr | -13 hr | -16 hr | N/A |
| 2 × 100 bp | ~19 hr | -19 hr | -25 hr | -36 hr |
| 2 × 150 bp | ~25 hr | ~25 hr | -36 hr | -44 hr |
| 2 × 250 bp | ~38 hr | N/A | N/A | N/A |

Quality scores

| | |
|---|---|
| 2 × 50 bp | ≥85% of bases higher than Q30 at 2 × 50 bp |
| 2 × 100 bp | ≥80% of bases higher than Q30 at 2 × 100 bp |
| 2 × 150 bp | ≥75% of bases higher than Q30 at 2 × 150 bp |
| 2 × 250 bp | ≥75% of bases higher than Q30 at 2 × 250 bp |

Run time

| | | | | |
|---|---|---|---|---|
| 2 × 50 bp | ~13 hr | ~13 hr | ~16 hr | ~N/A |
| 2 × 100 bp | ~19 hr | ~19 hr | ~25 hr | ~36 hr |
| 2 × 150 bp | ~25 hr | ~25 hr | ~36 hr | ~44 hr |
| 2 × 250 bp | ~38 hr | N/A | N/A | N/A |

Estimated Sample Throughputs

| | | | | |
|---|---|---|---|---|
| Human | ~4 | ~8 | ~20 | ~48 |
| Genomes per | | | | |
| Run | | | | |
| Exomes | ~40 | ~80 | ~200 | ~500 |
| per Run | | | | |
| Transcript- | ~32 | ~64 | ~164 | ~400 |
| omes | | | | |
| per Run | | | | |

Output and read number specifications in Table 1 are based on a single flow cell using an Illumina Phix control library at supported cluster densities. The sequencer can run one or two flow cells simultaneously. With regard to the quality scores, performance may vary based on library type and quality, insert size, loading concentration, and other experimental factors. Run times are based on running two flow cells of the same type. Starting two different flow cells will impact run time. All sample throughputs are estimates and are based on dual flow cell rules. Human genomes assumes >120 Gb of data per sample to achieve 30× genome coverage. Exome assumes ˜8 Gb/100×. Transcriptomes assume >=50M reads. Throughput may bary base on the library preparation kit used.

In some embodiments, one or more flow cell characteristics may be added or removed from the characteristics considered by the system. For example, if all sequencers in an exemplary laboratory support only four flow cells, such as the SP, S1, S2, and S4 of the Novaseq 6000 System, then flow cells of other systems may be excluded from the allocation. In another example, only the types of sequencers available at the time of allocation may have their respective flow cells included in the allocation determination.

In some embodiments, other sequencing systems may have other combinations of flow cell lanes with their own varying number of flow cell wells and the system may balance sample allocation between the differing sequencing systems and flow cells available at each pool generation.

In some embodiments, the sample data database 120 can include one or more flow cell sample configurations and/or pools generated by the sample allocation application 132 based on sample data included in the sample data database 120 and flow cell data included in the flow cell data database 124.

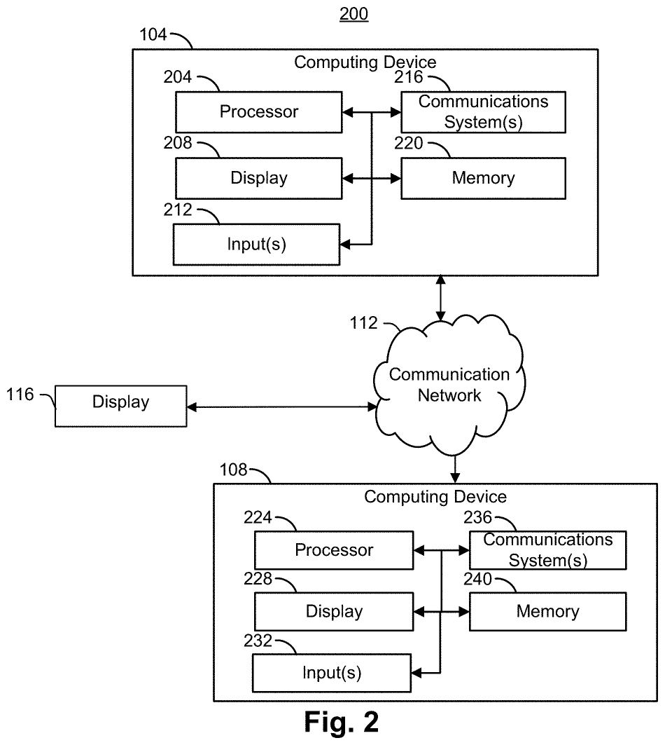

FIG. 2 shows an example 200 of hardware that can be used in some embodiments of the system 100. The computing device 104 can include a processor 204, a display 208, an input 212, a communication system 216, and a memory 220. The processor 204 can be any suitable hardware processor or combination of processors, such as a central processing unit (“CPU”), a graphics processing unit (“GPU”), etc., which can execute a program, which can include the process 800 described below.

In some embodiments, the display 208 can present a graphical user interface. In some embodiments, the display 208 can be implemented using any suitable display devices, such as a computer monitor, a touchscreen, a television, etc. In some embodiments, the inputs 212 of the computing device 104 can include indicators, sensors, actuatable buttons, a keyboard, a mouse, a graphical user interface, a touch-screen display, etc.

In some embodiments, the communication system 216 can include any suitable hardware, firmware, and/or software for communicating with the other systems, over any suitable communication networks. For example, the communication system 216 can include one or more transceivers, one or more communication chips and/or chip sets, etc. In a more particular example, communication system 216 can include hardware, firmware, and/or software that can be used to establish a coaxial connection, a fiber optic connection, an Ethernet connection, a USB connection, a Wi-Fi connection, a Bluetooth connection, a cellular connection, etc. In some embodiments, the communication system 216 allows the computing device 104 to communicate with the secondary computing device 108.

In some embodiments, the memory 220 can include any suitable storage device or devices that can be used to store instructions, values, etc., that can be used, for example, by the processor 204 to present content using display 208, to communicate with the secondary computing device 108 via communications system(s) 216, etc. The memory 220 can include any suitable volatile memory, non-volatile memory, storage, or any suitable combination thereof. For example, the memory 220 can include RAM, ROM, EEPROM, one or more flash drives, one or more hard disks, one or more solid state drives, one or more optical drives, etc. In some embodiments, the memory 220 can have encoded thereon a computer program for controlling operation of computing device 104 (or secondary computing device 108). In such embodiments, the processor 204 can execute at least a portion of the computer program to present content (e.g., user interfaces, images, graphics, tables, reports, etc.), receive content from the secondary computing device 108, transmit information to the secondary computing device 108, etc.

The secondary computing device 108 can include a processor 224, a display 228, an input 232, a communication system 236, and a memory 240. The processor 224 can be any suitable hardware processor or combination of processors, such as a central processing unit (“CPU”), a graphics processing unit (“GPU”), etc., which can execute a program, which can include the processes described below.

In some embodiments, the display 228 can present a graphical user interface. In some embodiments, the display 228 can be implemented using any suitable display devices, such as a computer monitor, a touchscreen, a television, etc. In some embodiments, the inputs 232 of the secondary computing device 108 can include indicators, sensors, actuatable buttons, a keyboard, a mouse, a graphical user interface, a touch-screen display, etc.

In some embodiments, the communication system 236 can include any suitable hardware, firmware, and/or software for communicating with the other systems, over any suitable communication networks. For example, the communication system 236 can include one or more transceivers, one or more communication chips and/or chip sets, etc. In a more particular example, communication system 236 can include hardware, firmware, and/or software that can be used to establish a coaxial connection, a fiber optic connection, an Ethernet connection, a USB connection, a Wi-Fi connection, a Bluetooth connection, a cellular connection, etc. In some embodiments, the communication system 236 allows the secondary computing device 108 to communicate with the computing device 104.

In some embodiments, the memory 240 can include any suitable storage device or devices that can be used to store instructions, values, etc., that can be used, for example, by the processor 224 to present content using display 228, to communicate with the computing device 104 via communications system(s) 236, etc. The memory 240 can include any suitable volatile memory, non-volatile memory, storage, or any suitable combination thereof. For example, the memory 240 can include RAM, ROM, EEPROM, one or more flash drives, one or more hard disks, one or more solid state drives, one or more optical drives, etc. In some embodiments, the memory 240 can have encoded thereon a computer program for controlling operation of secondary computing device 108 (or computing device 104). In such embodiments, the processor 224 can execute at least a portion of the computer program to present content (e.g., user interfaces, images, graphics, tables, reports, etc.), receive content from the computing device 104, transmit information to the computing device 104, etc.

The display 116 can be a computer display, a television monitor, a projector, or other suitable displays.

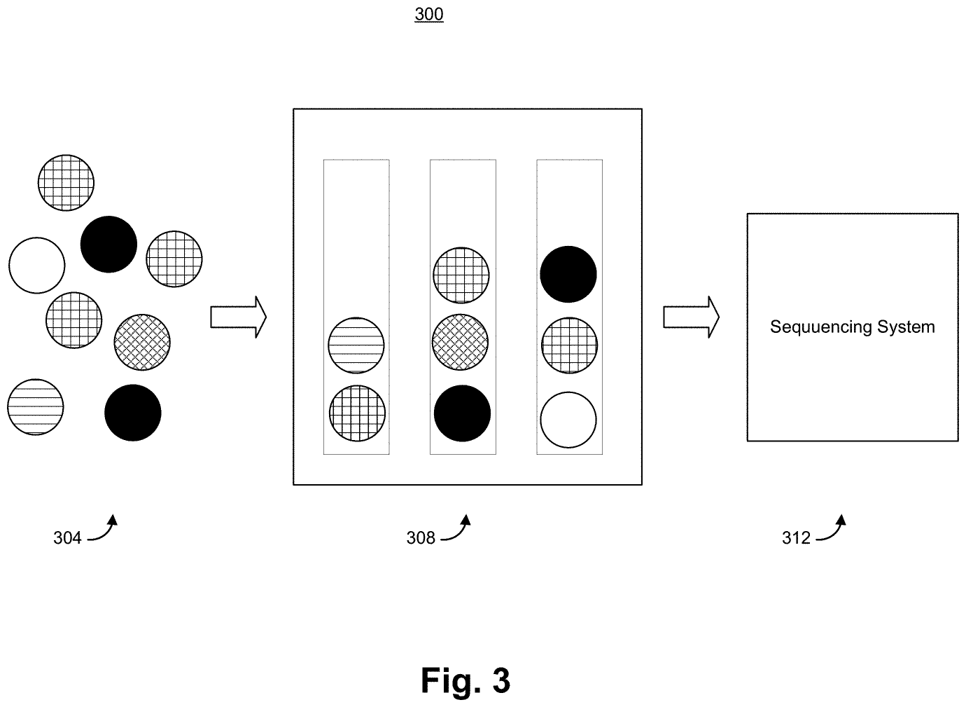

Referring now to FIG. 3, an exemplary, simplified embodiment of a flow 300 for sequencing samples in one or more flow cells is shown. The flow can include receiving samples 304, allocating the samples 308, and sequencing the allocated samples 312. The samples can include genetic material sampled from one or more regions (e.g., tumors, organs, etc.) of one or more patients. In some embodiments, each sample can include DNA and/or RNA derived from cells, cell-free DNA, bacteria, viruses, animals, and/or humans. The allocating step 308 includes the steps of selecting the samples to be sequenced, selecting which flow cells to use, and determining how to arrange samples in the flow cells, although the methods disclosed herein recognize that, while each of those sub-steps may be governed by its own rules or factors, there also is overlap between those factors or rules, e.g., the process of selecting which samples to sequence may depend on the number and/or type of flow cells that are available. Similarly, the process of selecting which flow cells to use may depend on the types of samples that are available to be sequenced.

The placement of samples into flow cells, pools and lanes can determine the overall quality of the sequencing outcome. Some sample properties that can be considered in choosing arrangements of samples are a mass of genetic material available, a type of genetic material (e.g., RNA and/or DNA), a source tissue (e.g., solid tumor, blood or other), and/or a size of the genetic fragments (e.g., size in terms of base pairs) that constitute the sample. Other factors such as a turnaround time, whether or not a matching tumor/normal has been received, and/or sequencing priority can also be considered in placing samples. Once the samples to be sequenced have been selected and the flow cells to use also have been selected, the samples can be placed in one or more flow cells each including at least one lane.

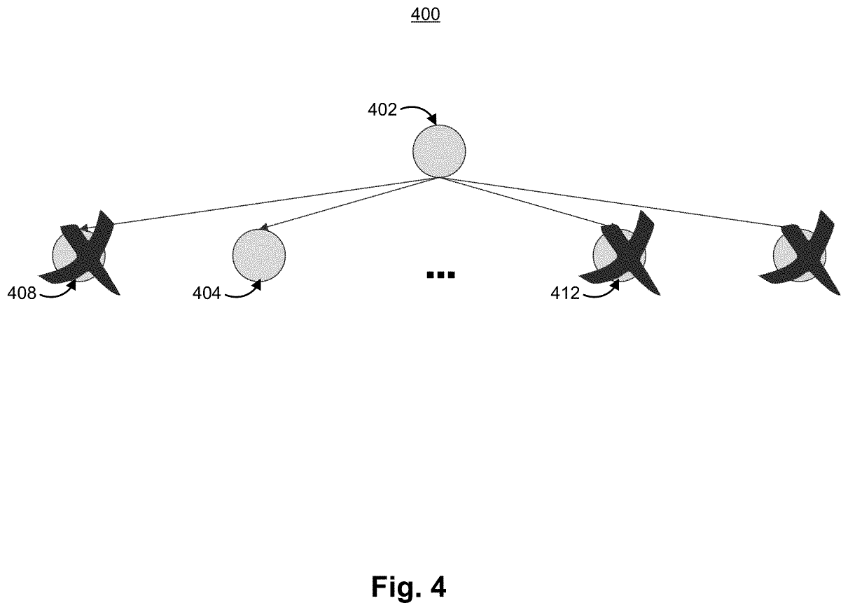

As it relates to the process of selecting which flow cells to use, FIG. 4 shows an exemplary search tree 400 that can be used to search for and/or select suitable groups of one or more flow cells to which a plurality of samples can be allocated. In some embodiments, each node included in the search tree 400 can include one or more flow cells or at least a portion of a flow cell group having one or more flow cells, each of the flow cells having a number of lanes, a minimum capacity, and a maximum capacity, where the minimum and maximum capacities depend on the sample types (tumors or normals, RNA or DNA, assay 1 vs assay 2, and/or other specimen delineators) being run on each lane. The minimum capacity and the maximum capacity can be how many samples need to be positioned in the flow cell and/or each lane in order to obtain proper sequencing. Implicitly, it can be assumed that there are an infinite number of flow cells and flow cell types available at a root node 402, but unnecessary solutions (e.g., more flow cells than samples) can be pruned at subsequent levels.

The tree can include a second level including a number of nodes generated based a number of flow cells that can be used. Specifically, out of all combinations of number of flow cells and flow cell types (e.g., in the root node 402), the second layer can be pruned by setting an upper bound to the practical number of flow cells that can be used. In some embodiments, the number of flow cells that can be used can be determined based on a number of sequencers available. For example, a node including more flow cells than the number of sequencers available can be pruned from the second layer, and expansion at the node can be terminated (e.g., no children nodes).

As noted above, each sample in the plurality of samples can be associated with a barcode, although the barcodes may not be unique as among all samples. When allocating the samples, barcodes with the same ID cannot be placed in the same lane in a flow cell in order to avoid a condition referred to as a “barcode clash.” In order to ensure that barcode clash does not occur, the tree 400 can be searched for nodes that include at least as many lanes as a maximum barcode clash. For example, if the plurality of samples includes four samples associated with the same barcode, a first node 404 in the second level of the tree 400 and including two flow cells with two lanes each (four total lanes) may be a valid node, while a second node 408 in the second level and including only one flow cell with one lane may be an invalid node. The tree 400 can be searched for nodes having a number of lanes at least as great as the maximum barcode clash or up to a predetermined amount above the maximum barcode clash (e.g., two lanes above the maximum barcode clash), which can prevent excess searching. Invalid nodes and their related flow cells may be disqualified from current sample allocation, as represented by the “X” through node 408 in FIG. 4.

Each node in the tree 400 can also include a minimum capacity and a maximum capacity equal to a sum of the minimum capacity(ies) of each flow cell included in the node and a sum of the maximum capacity(ies) of each flow cell included in the node, respectively. The tree 400 can be searched for nodes including a minimum capacity less than or equal to the number of samples in the plurality of samples and a maximum capacity greater than or equal to the number of samples in the plurality of samples. For example, if there are fifteen samples in the plurality of samples, and the first node 404 includes a minimum capacity of twelve and a maximum capacity of sixteen, the first node can be a valid node. To continue the example, a third node 412 having a minimum capacity of sixteen and a maximum capacity of twenty can be an invalid node. The third node 412 can be included in the second level of the tree 400. The search tree 400 can provide an efficient method to easily eliminate infeasible flow cell groups and identify feasible flow cell groups than may be used to allocate the plurality of samples.

In some embodiments, the tree 400 can include subsequent levels of nodes. Specifically, in some embodiments, a third level (not shown) can be generated based on flow cell types and lane types. Although flow cell types and lane types can be independent properties, in some embodiments each arrangement of lanes on each flow cell type may be considered as its own independent flow cell type. For example, flow cell types can be represented as “S1 flow cell with one lane of normals and one lane of tumors” or “S1 flow cell with two lanes of tumors”. If a node in the second layer of the tree 400 includes two flow cells, the tree 400 may include several hundred nodes at the third layer including a node including zero flow cells, another node including two flow cells of a given type and/or lane combination, yet another node including with mixed flow cell/lane types, and/or other permutations.

Just as nodes in the second layer may be pruned based on a first set of heuristics, nodes included in the third layer similarly may be pruned using a second set of heuristics. For example, in some embodiments, one heuristic can be verifying that there are enough positive controls to put one positive control on each tumor lane (e.g., a pathology requirement). Another heuristic can be verifying that there are enough lanes to run all clashing VIP samples (e.g., a business requirement). Yet another heuristic can be verifying that there are enough samples of each lane type to fill each lane to its minimum capacity (e.g., a bioinformatics requirement).

The third layer can be further generated by resolving clashing. For example, if there are two lanes total, amongst all flow cells, and three samples with the same barcode, all three samples cannot be sequenced. A process can determine that two samples can be run and one sample can be held. A number of total samples can be updated (e.g., one less sample due to the sample being removed), which becomes a portion of the data contained in the node. These nodes can be pruned by terminating flow cell arrangements.

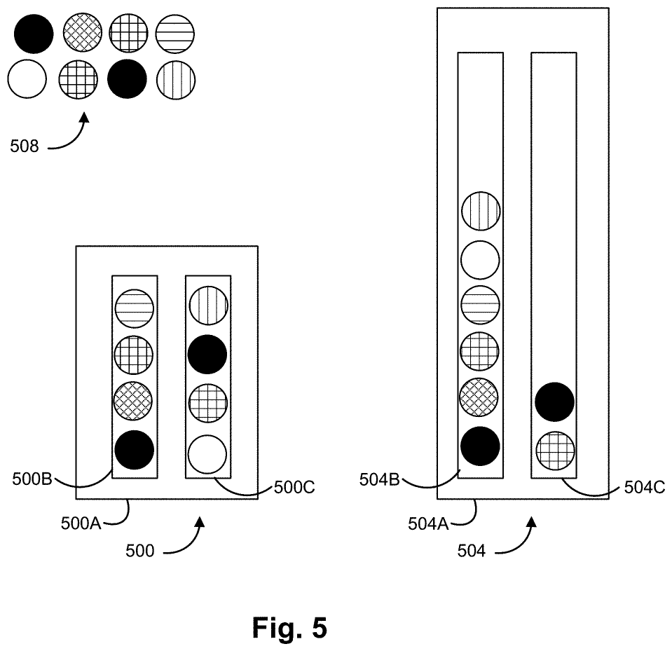

FIG. 5 shows an example of flow cell sample configurations. In particular, a first flow cell sample configuration 500 and a second flow cell sample configuration 504 are shown. A plurality of samples 508 including eight samples can be allocated in a valid fashion according to the first flow cell sample configuration 500 and in an invalid fashion according to the second flow cell sample configuration 504. Both the first flow cell sample configuration 500 and the second flow cell sample configuration 504 can include a flow cell group identified by searching the tree 400 in FIG. 4.

The plurality of samples 508 can include a first pair of samples associated with a first barcode, a second pair of samples associated with a second barcode, and four samples each associated with unique barcodes. The first flow cell sample configuration 500 can include a flow cell 500A including a first lane 500B and a second lane 500C. The flow cell 500A can include a minimum capacity of six samples and a maximum capacity of eight samples. One sample from each of the first pair of samples and the second pair of samples can be placed in each lane 500B, 500C, and the remaining samples included in the plurality of samples 500 can be placed in either lane. It is noted that at this stage in allocation, the specific location of any of the samples in either of the lanes 500B, 500C does not need to be known, just that the plurality of samples 508 can be arranged while avoiding barcode clash and satisfying the minimum capacity and the maximum capacity of the flow cell 500A.

The second flow cell sample configuration 504 can include a flow cell 504A including a first lane 504B and a second lane 504C. The flow cell 504A can include a minimum capacity of eight samples per lane and a maximum capacity of sixteen samples per lane. While the plurality of samples 508 satisfies the minimum lane capacity and maximum lane capacity requirements, and the flow cell 504A includes enough lanes to avoid barcode clash, there is no orientation that allows the plurality of samples 512 to be arranged and simultaneously satisfy the minimum lane capacity and the maximum lane capacity without barcode clash, and vice versa.

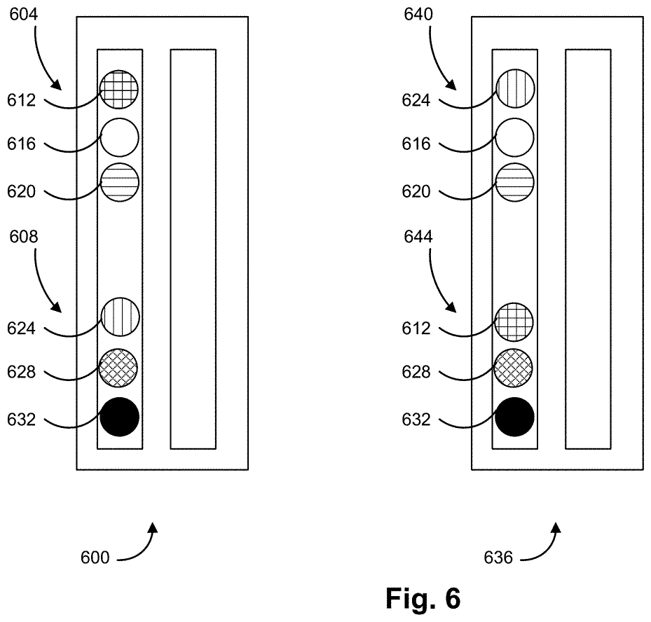

FIG. 6 shows an example of a first flow cell sample configuration 600 and a second flow cell sample configuration 636 each having multiple pools. Specifically, the first flow cell sample configuration 600 can include a first pool 604 and a second pool 608, and the second flow cell sample configuration 636 can include a first pool 640 and a second pool 640. Each of the pools 604, 608, 636, 640 can be generated by mixing (e.g., physically mixing) one or more samples in a liquid. Each of the first flow cell sample configuration 600 and a second flow cell sample configuration 636 can be valid flow cell sample configurations that satisfy flow cell requirements (e.g., capacity requirements) without barcode clashing. However, one of the first flow cell sample configuration 600 and a second flow cell sample configuration 636 may be preferable based on one or more constraints.

In the first flow cell sample configuration 600, the first pool 604 can include a first sample 612, a second sample 616, and a third sample 620, and the second pool 608 can include a fourth sample 624, a fifth sample 628, and a sixth sample 632. The first pool 604 and the second pool 608 can include samples chosen based on one or more constraints such as a mass balancing threshold (e.g., a maximum allowable difference in weight between samples in a pool), a turnaround time constraint (e.g., a hold cost of not running a sample based on a turnaround time associated with the sample), a maximum pool mass, a maximum lane mass, a maximum flow cell mass, a minimum pool mass, a minimum lane mass, a minimum flow cell mass, a prioritization level, and/or flow cell cost (e.g., a processing cost of running the sample). In some embodiments, one or more constraints can be used to generate scores for a number of pools, and final pools can be chosen from the highest scoring pools. In some embodiments, a prioritization level can be generated based on one or more directives. For example, a research sample may have a lower priority than a clinical sample. As another example, samples associated with a specific project (e.g., a prioritized project) may have a higher prioritization level than other “standard” projects.

In the second flow cell sample configuration 636, the first pool 640 can include the second sample 616, the third sample 620, and the fourth sample 624, and the second pool 644 can include the first sample 612, the fifth sample 628, and the sixth sample 632. In some configurations, a performance score can be calculated based on one or more constraints (e.g., compatibility scores and hold costs) for each of the pools in the first flow cell sample configuration 600 and the second flow cell sample configuration 636. The second flow cell sample configuration 636, and more specifically the first pool 640 and the second pool 644, may have better scores than the first flow cell sample configuration 600, and more specifically the first pool 604 and the second pool 608. The first flow cell sample configuration 600 can be chosen over the first flow cell sample configuration 600 to allocate a flow cell.

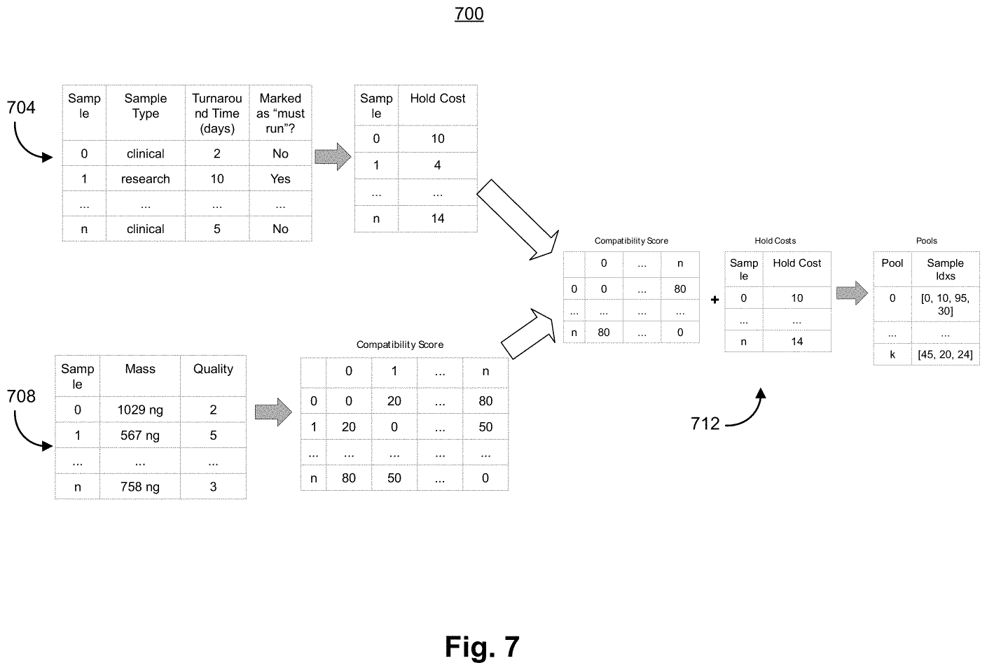

FIG. 7 shows an exemplary flow 700 for generating a plurality of pools based on a plurality of samples. The flow 700 can include calculating a hold cost 704 for each sample in the plurality of samples based on one or more sample characteristics. The one or more sample characteristics can include sample type, turnaround times, and/or a yes/no “must run” category. The flow 700 can include calculating a compatibility score for each pair of samples in the plurality of samples. The compatibility scores can be included in a matrix. The flow 700 can include generating the plurality of pools, each pool including at least a portion of the plurality of samples, based on the hold cost 704 for each sample and the compatibility scores.

In some embodiments, the compatibility scores and the hold costs can be provided to a greedy optimization algorithm using empirically tuned hyperparameters that balance short term optimality with the likelihood of obtaining a valid final result to pool samples in a way that minimizes the total cost of the run and maximizes the overall likelihood of obtaining valid results for each samples. For example, turnaround time depends on the type of sample (research prospective, research retrospective, clinical, etc.) as well as business requirements (e.g., a promised turnaround time). But in general, if a sample is exceeding a promised turnaround time, it may be preferable to run the sample more than a recently received sample. In some embodiments, this type of situation can be accounted for, e.g., with the algorithm “cost to not run=c1*exp(time-since-received/c2)” where c1 and c2 are constants that are hand tuned and vary based on the factors described above. In some embodiments, tumors and normals can be weighted differently because tumor results alone can have some value, but normals alone generally do not have value. All of the above factors can be accounted for adding all factors up to generate an “opportunity cost to not run this sample.”

In some embodiments, a loss score can be calculated for each pair of samples based on any predetermined pathology and/or research and development requirements. The loss score may function as a compatibility score similar to the scores depicted in FIG. 7. In this instance, the score may be normalized to be between 0 and 1 rather than 0 and 100, although the score alternatively could be between 0 and 100 as in FIG. 7 or between any other lower and upper bounds. In this case, if two samples are identical, the pair of samples can get a score of 1. Although there may be a multitude of factors that can be compared for each pair of samples, the method may designate one or more factors as more significant or gateway factors. For example, if one sample has a mass of 50 ng and the other sample has a mass of 1000 ng (e.g., a large difference in mass) the pair of samples can get a score of 0, independent of other factors. If the two samples have a mass of 500 ng and 650 ng, respectively, the pair of samples may receive a compatibility score between 0.25 and 0.75, depending on other factors like tissue type, size of the basepairs, etc.

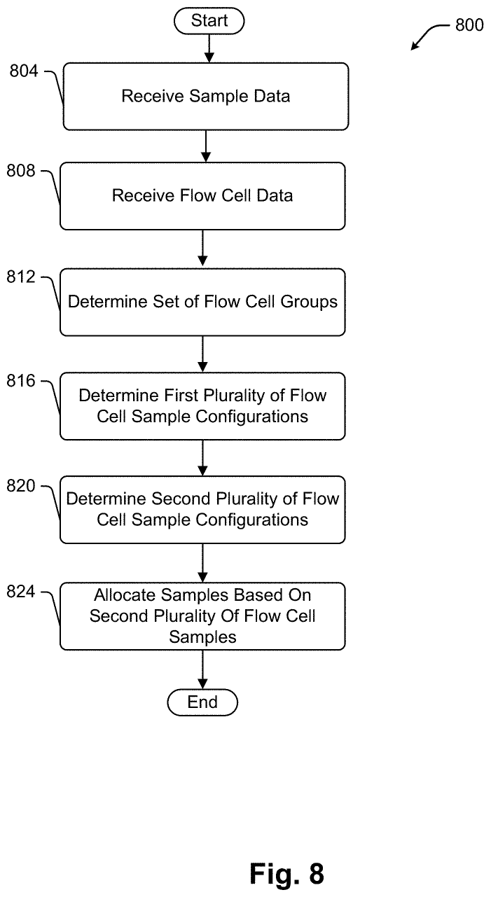

FIG. 8 shows an exemplary process 800 that can allocate samples in a flow cell based on sample data and flow cell data. The process 800 can be implemented as computer readable instructions on one or more memories or other non-transitory computer readable media, and executed by one or more processors in communication with the one or more memories or other media. In some embodiments, the process 800 can be implemented as computer readable instructions on the memory 220 and/or the memory 240 and executed by the processor 204 and/or the processor 224.

At 804, the process 800 can receive sample data. The sample data can include, for each sample included in a plurality of samples, a set of sample characteristics. In some embodiments, the sample characteristics can include a mass, a barcode, a source tissue type, an age, and/or a turn-around-time. In some embodiments, the set of sample characteristics can include a sample type that can be a tumor, a normal match, and/or a control.

At 808, the process 800 can receive flow cell data. The flow cell data can include, for each flow cell included in a plurality of flow cells, a set of flow cell characteristics. In some embodiments, the flow cell characteristics can include a maximum capacity, a minimum capacity, and/or a number of lanes. In some embodiments, the flow cell characteristics include an availability of a flow cell, and the process may include evaluating the availability of one or more flow cells. When a flow cell is unavailable, the process 800 may include delaying sequencing the unavailable flow cell until the unavailable flow cell becomes available. In some embodiments, the plurality of flow cells can include about twelve flow cells. In some embodiments, the plurality of flow cells can include Illumina flow cells.

At 812, the process 800 can determine a set of flow cell groups. In some embodiments, the process 800 can determine the set of flow cell groups based on the flow cell characteristics and a number of samples included in the flow cell samples. Each flow cell group included in the set of flow cell groups can include at least one flow cell. In some embodiments, the process 800 can search a tree (e.g., tree 400 in FIG. 4) to find suitable flow cell groups.

In some embodiments, the process 800 can generate a preliminary set of flow cell groups including unique combinations of one or more flow cells in the plurality of flow cells.

The process 800 can then determine a secondary set of flow cell groups based on a number of samples included in the plurality of samples. The secondary set of flow cell groups can include at least a portion of the preliminary set of flow cell groups. The process 800 can, for each flow cell group included in the secondary set of flow cell groups, determine that the maximum capacity of each flow cell included in the flow cell group is not exceeded by the number of samples, and determine that the minimum capacity of each flow cell included in the flow cell group is satisfied by the number of samples. The process 800 can also determine a maximum number of repeated barcodes (maximum barcode clash) based on the barcode associated with each sample in the plurality of samples. The process 800 can determine a tertiary set of flow cell groups by determining, for each flow cell group included in the secondary set of flow cell groups, that a sum of the number of lanes associated with each flow cell in the flow cell group is at least as great as the maximum number of repeated barcodes, the set of flow cell groups including the tertiary set of flow cell groups.

Once the process has determined the possible flow cell groups, then, at 816, it can determine a first plurality of flow cell sample configurations. In some embodiments, the process 800 can determine the first plurality of flow cell sample configurations based on the sample data and the flow cell data. In some embodiments, each flow cell sample configuration included in the first plurality of flow cell sample configurations can include a flow cell group included in the set of flow cell groups. In some embodiments, each flow cell sample configuration can be configured to house each sample included in the plurality of samples. For example, the first plurality of flow cell sample configurations can include the first flow cell sample configuration 500 but not the second flow cell sample configuration 504 in FIG. 5.

In some embodiments, the process 800 can determine to include a specific flow cell sample configuration in the first plurality of flow cell sample configurations by determining that the maximum capacity and the minimum capacity of each flow cell included in the specific flow cell sample configuration can be satisfied while keeping samples associated with equal barcodes in different lanes included in the specific flow cell sample configuration based on the number of lanes associated with each flow cell in the specific flow cell sample configuration and the barcode associated with each sample included in the plurality of samples.

In some embodiments, the process 800 can determine a processing cost for each flow cell sample configuration in the first plurality of flow cell sample configurations. The processing cost can be reflective of the processing power required to sequence a specific flow cell sample configuration. The processing cost can vary significantly between flow cell sample configurations. In some embodiments, the process 800 can rank the flow cell sample configurations by processing cost and/or filter out flow cell sample configurations above a predetermined processing cost threshold. The processing cost can vary based on sample type (e.g., a tumor, a normal match, and/or a control).

At 820, the process 800 can determine a second plurality of flow cell sample configurations. In some embodiments, the process 800 can determine the second plurality of flow cell sample configurations based on the sample data, the flow cell data, and at least one constraint. The at least one constraint can include a maximum pool mass, a maximum lane mass, a maximum flow cell mass, a minimum pool mass, a minimum lane mass, a minimum flow cell mass, a mass balancing threshold, a turnaround time constraint, prioritization level, and/or flow cell cost. The at least one constraint may include at least one soft constraint and/or at least one hard constraint.

In some embodiments, each flow cell sample configuration included in the second plurality of flow cell sample configurations can include a flow cell sample configuration included in the first plurality of flow cell sample configurations. In some embodiments, the process 800 can generate a plurality of hold costs based on a turnaround time associated with each sample included in the plurality of samples. Each sample included in the plurality of samples can be associated with a hold cost included in the plurality of hold costs. The process 800 can determine the second plurality of flow cell sample configurations based on the plurality of hold costs.

In some embodiments, the process 800 can generate a plurality of compatibility scores based on the sample data. Each unique pair of samples included in the second plurality of flow cell samples can be associated with a compatibility score included in the plurality of compatibility scores. In some embodiments, the process 800 can generate a plurality of hold costs based on the turnaround time associated with each sample included in the plurality of samples. Each hold cost included in the plurality of hold costs can be associated with a sample included in the plurality of samples. The process 800 can generate a plurality of pools based on the plurality of compatibility scores and the plurality of hold costs (e.g., using the flow 700 in FIG. 7), each pool included in the plurality of pools including at least one sample included in the plurality of samples.

In some embodiments, the process 800 can generate a plurality of preliminary flow cell sample configurations based on at least a portion of the plurality of pools and the at least one constraint, and second plurality of flow cell sample configurations can include at least a portion of the plurality of preliminary flow cell sample configurations.

In some embodiments, the process 800 can generate a first set of pools based the sample data, the flow cell data, and a first constraint included in the at least one constraint. Each pool included in the first set of pools can include at least one sample included in the plurality of samples. The process 800 can generate a second set of pools including a subset of the first set of pools based on the sample data, the flow cell data, and a second constraint included in the at least one constraint. The process 800 can generate the second plurality of flow cell sample configurations based on the second set of pools. For example, the process 800 can identify a first set of pools that satisfy a minimum pool mass constraint, and then identify pools that also satisfy a mass balancing threshold constraint.

In some embodiments, the process 800 can generate pools with greedy optimization. The process 800 can place a first sample based on a constraint (e.g., place a sample with a highest hold cost for a hold cost constraint), then place a second sample (e.g., place a sample with the second highest hold cost for the hold cost constraint), and test for compatibility between the samples and the flow cell for all pools compatibility. In some embodiments, the process 800 can generate a matrix including, for each sample included in the plurality of samples, a pool location, a lane location, a flow cell location, and/or holdout yes/no category. It is noted that the process 800 may leave out one or more samples included in the plurality of samples from inclusion in the second plurality of flow cell sample configurations.

In some embodiments, the one or more constraints can be selected to balance mass (fragmented DNA) of samples for each pool, balance a correct number of reads with a highest number of samples, and/or reduce turnaround time. In some embodiments, the process 800 can ensure that the pools have been formed such that a single patient's specimen aren't intermixed in the same pool.

At 824, the process 800 can allocate samples based on the second plurality of flow cell sample configurations. In some embodiments, the process 800 can output (e.g., visual instructions for a lab technician and/or machine readable instructions to a sequencing system) the second plurality of flow cell sample configurations, which can include one or more pools generated at 820.

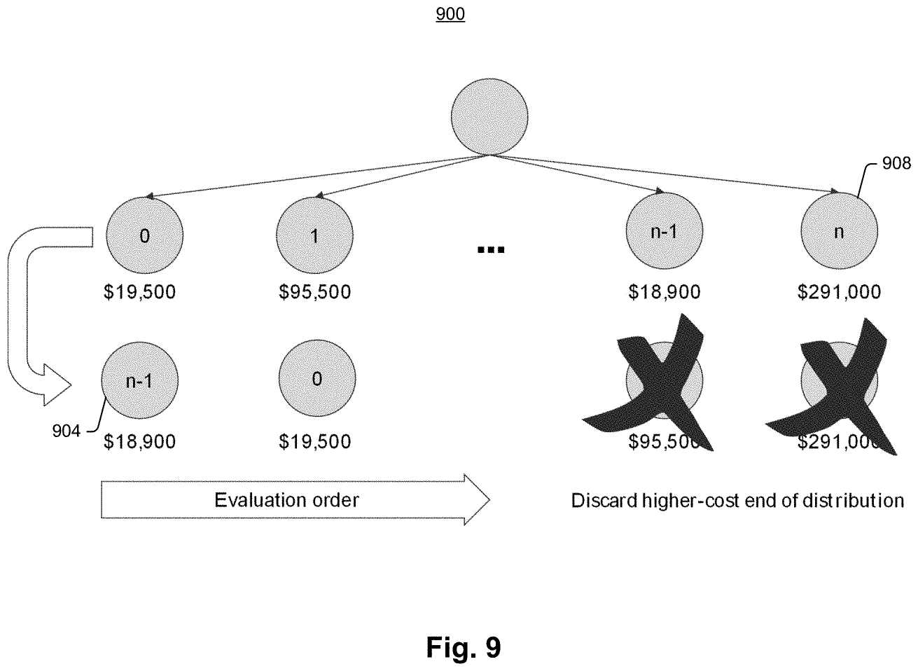

FIG. 9 shows an exemplary sorted tree 900. In some embodiments, the tree 900 can be generated at 816 in the process 800 in FIG. 8. As described above, in some embodiments, the process 800 can determine a processing cost for each flow cell sample configuration in the first plurality of flow cell sample configurations. The processing cost can be reflective of the processing power required to sequence a specific flow cell sample configurations and/or other cost factors. Thus, while the cost in FIG. 9 is represented in terms of dollars, it should be understood that that cost also could be expressed in terms of an amount of memory or processing time estimated to sequence the flow sample configuration(s), an estimated energy consumption to sequence the flow sample configuration, etc. The processing cost can vary significantly between flow cell sample configurations. In some embodiments, the process 800 can rank the flow cell sample configurations by processing cost and/or filter out flow cell sample configurations above a predetermined processing cost threshold. The processing cost can vary based on sample type (e.g., a tumor, a normal match, and/or a control). The tree 900 can include a least expensive node 904 and a most expensive node 908. The most expensive node 908 may get filtered out by the process 800, while the least expensive node 904 may be retained by the process 800. In testing, it has been found that the best solution can be found in the five least expensive nodes in the tree 900 the majority of the time.

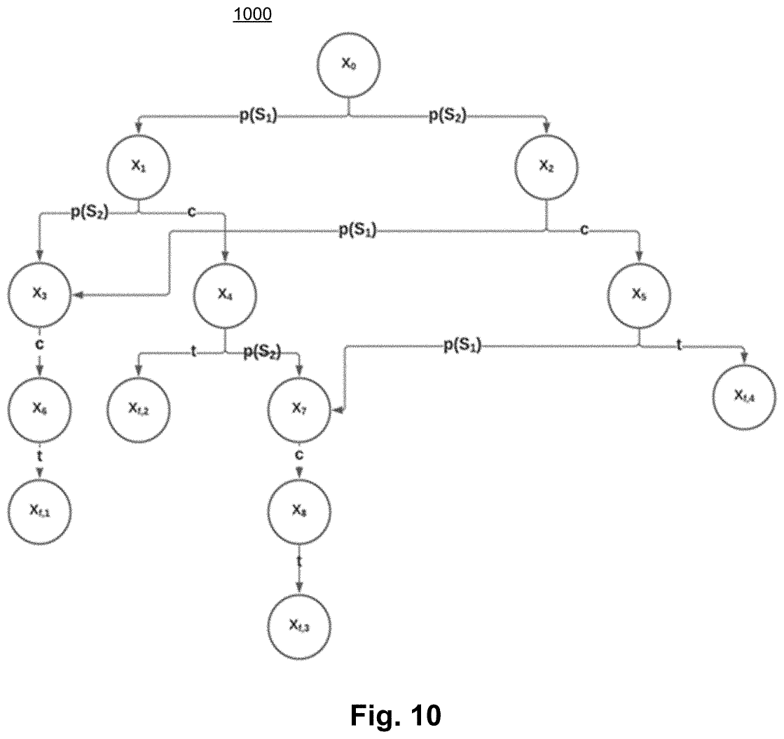

Referring to FIG. 10, another exemplary search tree 1000 is shown. The search tree 1000 can be used to aid in sequencing extracted, normalized and barcoded genetic material on certain sequencers, such as Illumina sequencers. First, samples can be pooled by combining several samples of similar mass/quality into one physical asset. Second, groups of pools can be placed onto flow cell lanes. Finally, entire flow cells layouts can be formed. Flow cells can be a minimum processing unit handled by sequencers. One end goal of pool planning can be assigning samples in an unordered set of samples to a pool within a lane within a flow cell, and by proxy selecting what flow cells will be run.

Table 2 below shows an exemplary sample queue (input). Table 3 below shows an exemplary plan (output). Table 4 below provides examples of flow cell types.

TABLE 2

| Asset | | | |
|---|---|---|---|
| ID | Mass | Quality | Barcode |
| 1234 | 200 ng | 4 | 1 |
| 4123 | 600 ng | 3 | 2 |
| 3412 | 250 ng | 5 | 1 |

TABLE 3

| Asset | Flow | | |
|---|---|---|---|
| ID | cell | Lane | Pool |
| 1234 | 1 | 1 | 1 |
| 4123 | 1 | 1 | 1 |
| 3412 | 2 | 1 | 1 |

TABLE 4

| Flow | |
|---|---|
| cell | Type |
| 1 | S1 xP |
| 2 | S4 Standard |

Flow cell planning can be referred to as “hyb planning” or “pool planning” and comprises planning out an entire sequencing run. Thus, a hyb script/planner can be understood as a function that maps from a sample queue to a sequencing plan. The sequencing plan can have an impact on turnaround time (TAT), costs, and validity of sequencing.

In some embodiments, this complex problem can be approximated as a Markov Decision Process, and more specifically a planning problem and/or resource allocation problem. Generally, the problem comprises making sequential decisions (selecting samples, selecting flow cells, putting samples on lanes, putting samples in pools, etc.) in an order that (ideally) moves from a starting point to a valid (and optimal) solution.

### Decomposing the Problem

Although decisions made at any stage of the process can affect the outcome (that is, how samples are arranged in pools can determine the validity of a choice of flow cells), the relationship is much stronger in one direction than another.

As described above, this problem can be decomposed into three distinct problems: selection of flow cells; selection of what samples to put in each lane of a flow cell; and selection of what pool within a lane to put each sample from that lane into.

### Flow Cell Selection

An example of flow cell planning will now be described. In this example, there are four types of flow cells: SP, S1, S2 and S4 in order of increasing capacity/cost. A planning system can utilize ˜4 sequencers at any given time to sequence the flow cells. Each flow cell type can be run with mixed lanes (first assay configuration) or with each lane being treated individually (second assay configuration). The first assay configuration can be run having any given lane with normals or tumors. Thus, there are ˜12 different flow cell types, and the sequencers can run anywhere between 0 and ˜4 of each type. In short, there are ˜16 million possible different flow cell configurations that can be considered.

In order to reduce the number of possible configurations, the system may apply one or more heuristics to rule out many of them. For example, if there are one hundred samples it does not make sense to run 4 S4 flow cells because a minimum sample loading required to get valid sequencing results cannot be obtained. Additionally, the system may evaluate the samples to determine whether multiple related samples (as determined, e.g., by whether those samples have the same barcode or other identifier) have higher priority than other samples. In addition to selecting those samples for analysis prior to selecting other samples, the system may discard flow cell configurations for those samples that have a single lane or a smaller number of lanes than there are high priority samples, because those flow cells would not be able to run all of those high priority samples. Other variables such as a maximum number of samples, a mass closeness, other factors as described herein, etc., may be used to determine this heuristic.

Applying this sort of heuristic pruning generally allows the number of flow cell configurations that must be considered to be reduced to thousands or hundreds.

### Lane Selection

After the number of flow cell configurations has been pruned using the first set of (flow cell configuration) heuristics, the system may apply a second set of heuristics to reduce the number of flow cell configurations further.

Simple heuristics for this step are harder to discern than for flow cell selection, such that the heuristics may be more computationally complex. For example, it generally has been found that a lane selection that starts with the highest degree of clashing samples (samples whose barcode is repeated the most times) leads to the best results.

For example, for a barcode 1234, there may be two samples that share the same barcode. There also may be two lanes, with one lane having higher capacity than the other. If one of the two samples is placed on each lane, it is guaranteed that there is no barcode duplication within lanes. On the other hand, if the samples are not placed at the same time, the planning process would have to undertake the more computationally intensive process of checking all other samples in a lane to see if a sample with that barcode exists. To avoid this, the planning process can place the samples at the same time. If the samples are placed at the beginning, when both lanes still have space, both samples can be appropriately placed. If the lane selection heuristic were to wait until one lane is full, only one sample can be placed. Thus, one lane selection heuristic may comprise identifying all samples that share at least one barcode or other identifier with at least one other sample and then placing all of those shared samples into lanes prior to placing any other samples.

In another example, given a flow cell configuration, the system may create a set of lanes for each sample type, for example, tumors/positives together on a lane and normals separately. Different assay configurations may configure lanes differently. Then for the set of all lanes that accept tumors and positive controls, a planning process can decide which samples to put on which lanes. This can depend on two factors, e.g: the value of running the samples (clinical samples can be prioritized over research samples, high TAT samples over low TAT samples, etc.), and the compatibility of samples (samples with the same barcode cannot be mixed within a lane, and all samples on a lane must form valid pools (otherwise, they cannot be run on that lane).

For example, the planning process may result in assay configurations that avoid a configuration such that all samples are run (good on the value/cost side) but low quality pools may be created and may reduce the quality of sequencing results for samples in the low quality pools.

The system may employ still other lane selection heuristics, such as starting with low mass and/or STAT samples. Unlike the other lane selection heuristics already described, the impact of any choice for these heuristics cannot be known until a final plan is created. In other words, these heuristics may be merely greedy choices of tree traversal.

### Pool Selection

Increasing still further in computational complexity, given a set of samples on a lane, pools (groups) of these samples can be created. Pools of samples of similar characteristics are generally better. Conversely, if the samples are too dissimilar, they can form a categorically invalid pool (e.g., a pool where the probability of sequencing failure is high enough that it would be more preferable to not even run those samples in that configuration). Generally larger (more samples per pool) are better, but not at the cost of creating low quality pools.

In one instance, heuristics may be used to greedily traverse the state space and select branches that generally lead to better outcomes. For example, low mass samples may be harder to match up, so the system may start with low mass samples by taking a sample, seeding a pool, and then trying to fill that pool with the most similar samples that exist amongst the remaining samples. One potential downside of this approach is that making a pool at one point in time affects the ability to make later pools. In particular, making the best pool now may be detrimental to future pools, or can even leave subsequent samples with no match. Thus, one alternative pool selection technique that may be applied is formal framing, as discussed as follows.

### Formal Framing

The pool selection process can be the most computationally intensive part and constitutes the “tight loop” of the entire process. This pool selection process can be framed as a non-abstract planning problem. There is an initial state (no pools, all of the samples are available to be pooled) and a final state (each sample has been either pooled or marked as not-poolable/not to be run), and the pool selection process can move between states by taking actions.

Traversal of the search tree 1000 will now be described. The initial state (X₀) may include the unordered queue of samples (Z={S₁, S₂, . . . , Sₙ}), along with an empty set of sets that represent the pools (P={P₁, P₂, . . . , Pₙ}, where Pᵢ may be {S₁, S₂, S₃}, for example). Together, these form an initial world state. The planning process can execute a sequence of actions that transfer our initial world state X₀ to a new world state Xᵢ and, which may or may not be a valid final state X_{f}. Generally, the planning process can only estimate the cost/reward of intermediary states (e.g., by rewarding running samples and not holding them), but the final reward/cost cannot be known until a final state is reached (since putting S₁ in P₁ may be detrimental to Pₙ even if it is the best match for P₁; this is a suitable short-term/long-term tradeoff).

There are generally three types of actions the planning process can perform. A first action can be placing a sample from the queue into the current pool (action p(S) in the search tree 1000, where S is the sample from Z the action is being applied to). The first action is only valid if the current pool is smaller than the max pool size (4 for a first assay, 8 for a second assay). A second action can be to close/complete the current pool and start a new one (action c in the search tree 1000). This action is only valid if the pool is above the minimum pool size (1 sample, 4 samples). A third action can be to terminate the current plan (action tin the search tree 1000), e.g., run the current pools and do not run any samples still in Z. This action may be valid if there are no open pools (e.g., if the preceding action was c).

The planning process can find a final solution state when the plan is terminated (last action was t) and/or a dead-end state is hit. Reasons the planning process may hit a dead-end state can include that the last closed pool that was made is invalid, the current in-progress pool cannot be made valid (it is not trivial to determine if a pool is “unsalvageable,” although certain underestimating heuristics can be applied to determine this (for example, a heuristic may be set that never marks a possibly valid pool as invalid, but may mark an invalid pool as valid)), and/or that there are not enough samples in a queue to bring the current pool up to the minimum pool capacity. Combining these elements, the problem can be represented by a search tree 1000 where every level of the tree is an action.

In the search tree 1000, X₀=(Z₀, P₀) with Z₀={1, 2} and P₀={ }, and: X_{f,1}=({ }, {{1, 2}}), X_{f,2}=({2}, {{1}}), X_{f,3}=({ }, {{1}, {2}}), and X_{f,4}=({1}, {{2}}).

A property of the search tree 1000 is highlighted here: since this is a Markov Decision Process where actions depend on the current state and different chains of actions may arrive at the same state. Recognizing and optimizing for this greatly reduces the memory and computation required to traverse the graph. For example, failing to recognize this would result in traversing from X₃ to X_{f,1} twice (albeit with different state names/identities), e.g., from X₀ through X₁ to X₃ and from X₀ through X₂ to X₃. Additionally, note that since model P is modeled as a set of sets, the chains of actions p(S₁)→c→p(S₂)→c→t and p(S₂)→c→p(S₁)→c→t result in the same final state since the order of the pools in P does not matter. The same applies to p(S₁)→p(S₂) and p(S₂)→p(S₁). Implementing the graph as a hashmap-based adjacency list enables an improvement over a purely object-oriented approach since nodes with the same hash will naturally coalesce paths and reduce wasteful decision pathing.

Within this framework, the overall task can be reduced to finding the sequence of actions that can be used to traverse this state space graph and arrive at an optimal solution. It is contemplated that it may be possible to expand this approach to also determine what lanes to put samples into by holding multiple sets of open/closed pools (one for each lane) and multiple sets of sets of pools (one for each lane). Depending on the shape of the search tree, this may be faster or slower than sequential decision making. This could be expanded further to select flow cells, although at that point it is likely that using sequential heuristics and dividing the problem as described above would prove more tractable than blowing up the branching factor of this framework by orders of magnitude.

As presented, the branching factor is in the order of approximately a number of samples in the queue, highlighting the significant drawbacks that a conventional system not applying one or more of the techniques experiences, particularly in high throughput applications. In particular, assuming a queue has 300 samples (a rough average for a first assay configuration), this problem has a worst-case brute force computational cost (if each action we take is making a pool with a single sample) in the order of ˜300!.

In some embodiments, flow cell planning can include generating millions of flow cell permutations, pruning the permutations using heuristics like capacity and ability to accommodate clashing, iteratively exploring all permutations of sample/lane combinations, and creating a full pooling plan for each lane on every iteration. The pooling plans are roughly a greedy iteration of the search tree 1000: instead of trying all possible actions, an “optimal” action is picked based on heuristics. In brief, we look for the most similar sample in the queue to the samples in the current pool. If adding it to the pool improves a “pool score”, then the sample can be added. Otherwise we start a new pool with it. Once all samples are in pools, we go through the pools and remove any invalid pools and mark those samples as held.

### Defining a Desired Outcome

In some embodiments, the desired outcome for developing an effective solution to flow cell planning can be defined by one or a combination of cost, TAT, and validity of sequencing results. It is desirable to minimize the cost (e.g., how much is spent on consumables (flow cells), how much memory and/or processing power is required to allocate and/or evaluate the samples) and the TAT while maximizing and/or maintaining high level of validity of the sequencing results. In particular, it is not desirable to compromise sequencing results for any cost.

TAT can be expressed in terms of a cost value (even if it is only a proxy), and so TAT and cost can be combined into a single criteria by assigning a “value” to each sample. This still leaves two often contrary fronts to optimize on.

One technique to define pool quality is by declaratively listing what should be valid and not. For example:

S1=(mass=200, quality=low, source tissue=ffpe)
S2=(mass=200, quality=low, source tissue=blood)
S3=(mass=200, quality=high, source tissue=ffpe)
{S1, S1, S1} MUST BE INVALID//not enough mass/samples
{S1, S1, S1, S1} MUST BE VALID//a full pool of low mass samples
{S1, S1, S1, 52} MUST BE INVALID//cannot mix ffpe and blood samples
{S1, S1, S1, 53} MUST BE INVALID//cannot mix low and high quality samples

For this example, quality may be determined using one or more factors such as tumor percentage, cell age and/or degeneration, biological quality of the specimen, etc. One or more of these factors may be used and/or stressed based on a type or quality of output needed for a specific purpose.

These preferences can then be encoded as a binary outcome and the pools can be transformed into features. Feeding this into a learning model has shown great success. In particular, boosted decision tree-based models like those offered by XGBoost are particularly well suited for this task.

The problem of comparing two pools (for example {S1, S1, S1, S1} vs. {S2, S2, S2, S2}) is a complicated matter that is made even more complicated in high throughput situations. One option is to try to assign a numerical score/value to each pool. This is very difficult in high throughput applications, however, because assigning scores to thousands of pools and having them all rank in the desired order is largely intractable.

Another option is to compute the weighted value of a pool, using the model trained for valid/invalid described above, where that model is Bayesian in order to generate probabilities rather than binary options. A validity model can determine what the probability of a pool being valid is, and then weight that value (as in the sum of the estimated cost value of the running the samples) of the pool with the probability that it is valid. In this way, the planning process can compare a mediocre pool with valuable samples to a great pool with non-valuable samples. This solution is particularly useful because it folds the cost and pool quality fronts into a single front while keeping the heuristic comparatively simple and rooted in real-world logic.

In one example, machine learning may be implemented to rank pools according to one or more metrics. Pairwise preferences between pools are fed into a model, and the model learns to rank them not only in pairs but also in larger groups. Possible machine learning ranking models that may be employed include, but are not limited to, RankBoost, RankNet, LambdaRank, Listnet, RankGP, LambdaSMART/LambdaMART, BayesRank, XGBoost, etc.

Overall, it is desirable to be able to assign numerical fitness scores to a given outcome. To do this, the pareto front efficiency tradeoff need to be resolved and techniques of encoding existing preferences into deterministic scoring models can be developed. In some embodiments, the deterministic scoring models can include machine learning models. In some embodiments, the machine learning models can be trained using reinforcement learning. In some embodiments, the machine learning models can be trained using reinforcement learning methods such as Markov Decision Process methods. In some embodiments, the machine learning models can be trained using reinforcement learning methods such as Monte Carlo methods.

The planning problem is largely amenable to solutions based on Monte Carlo tree search (MCTS) with learned selection and/or simulation functions. A well-known use of this approach is AlphaZero, although related and perhaps more generally applicable approaches exist, for example Aleph Star or MuZero. These approaches are particularly applicable to the planning problem because there is sufficient input data and simulations to train on are (relatively) cheap.

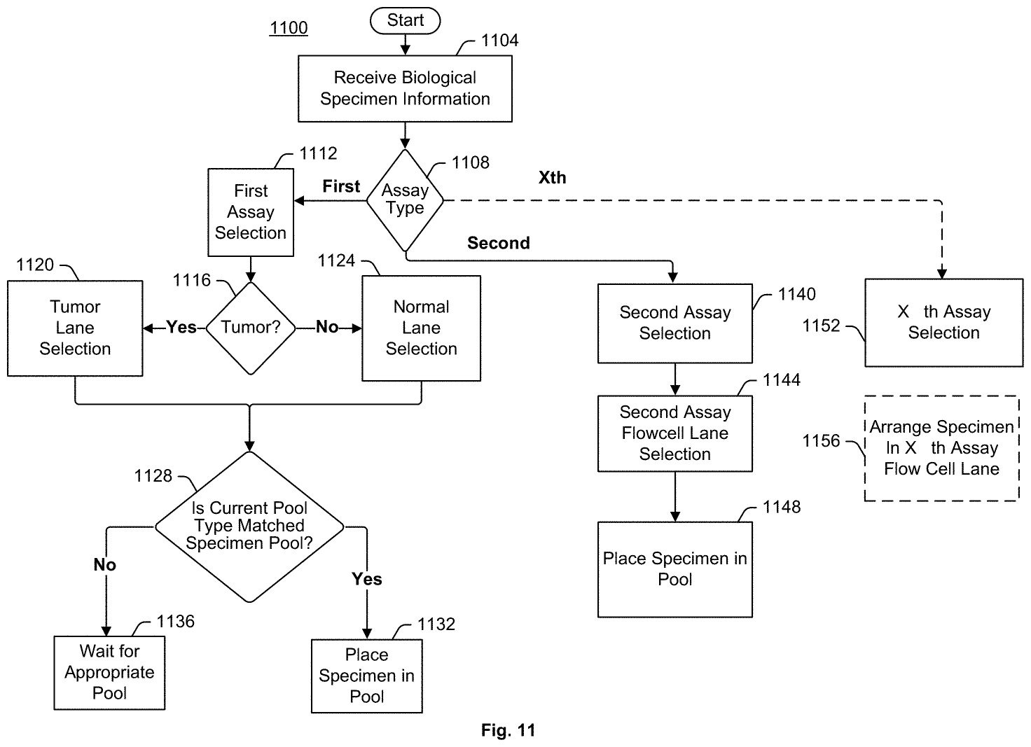

FIG. 11 is an exemplary process 1100 for implementing a specimen mapping system and/or a sequencing system. The process 1100 can be implemented as computer readable instructions on one or more memories or other non-transitory computer readable media, and executed by one or more processors in communication with the one or more memories or other media. In some embodiments, the process 1100 can be implemented as computer readable instructions on the memory 220 and/or the memory 240 and executed by the processor 204 and/or the processor 224. In some embodiments, the process 1100 can be executed by a sequencing system in order to arrange specimens in flow cells and sequence the specimens using the flow cells.

At 1104, the process 1100 can receive biological specimen information associated with a biological specimen. In some embodiments, the process 1100 can receive the biological specimen (e.g., in an automated specimen placement system). The specimen information can include at least a portion of the sample data described above. In some embodiments, the specimen information can include a mass, a barcode, a source tissue type, an age, and/or a turn-around-time. In some embodiments, the specimen information can include a sample type that can be a tumor, a normal match, and/or a control. In some embodiments, the specimen information can include a biopsy type that can be a liquid or a solid biopsy.

At 1108, the process 1100 can determine what assay type the biological specimen should be run with. In some embodiments, there can be a first assay configuration and a second assay configuration available. In some embodiments, the first assay configuration can be run with mixed lanes (e.g., liquid and/or solid specimens). In some embodiments, the second assay configuration can be run with each lane being treated individually (e.g., liquid only). In some embodiments, other assay limitations may be present. In some embodiments, the first assay configuration can be run having any given lane with normals or tumors (each lane only has normals or tumors). In some embodiments, there can be at least three assays available.

In some embodiments, each of the assays may target different portions of a genome, may be a targeted panel assay or a whole genome assay, may be an proprietary assay and/or a third-party assay, may be an assay approved by a different hospitals, medical institutions, and/or government agencies, and/or may use different sequencing methodologies altogether. In some embodiments, the flow cell(s) associated with the assays, and the corresponding optimization logic, may vary in complexity based on the assay selected. For example, the second assay may be used significantly less than the first assay and due to the reduced use can be sent straight to pooling with minimal computation due to the reduced number of samples. Generally, with higher throughput needed for an assay, the more complex and demanding the processing and optimization logic will be to handle it. It is appreciated that even with a relatively low number of specimens (e.g., five samples), the complexity of generating pools of specimens may be too great for a human to reasonably perform in their head, and computing systems may be required to implement the process 1100.

In some embodiments, the process 1100 can determine that only one assay configuration is suitable for the specimen (e.g., the specimen is a solid and there is only one assay configuration available for solids) and proceed to the appropriate assay selection. For example, the process 1100 can determine that the specimen is a solid and proceed to 1112. In same embodiments, if multiple assay configurations can be used with the specimen, the process 1100 can determine which assay is most applicable (e.g., which assay is associated with a lower cost flow cell, which assay Is more readily available, etc.) and proceed to that assay selection. For example, the process 1100 can determine that the specimen is a liquid sample and proceed to 1136. In some embodiments with more than two assays, the process 1100 can proceed to an Xth assay selection 1152 if that assay is the most applicable. However, it is appreciated that Xth assay selection 1152 is optional.

At 1112, the process 1100 can select a flow cell associated with the first assay configuration. In some embodiments, the process 1100 can receive flow cell data. The flow cell data can include a set of flow cell characteristics associated with a flow cell. In some embodiments, the flow cell characteristics can include a maximum capacity, a minimum capacity, and/or a number of lanes. In some embodiments, the flow cell can be an Illumina flow cell.

At 1116, if the specimen is a tumor sample, the process 1100 can proceed to 1120. At 1116, if the specimen is not a tumor sample, the process 1100 can proceed to 1124.

At 1120, the process 1100 can determine which lane is available to place the specimen. In particular, for one or more flow cells each having the set of flow cell characteristics, the process 1100 can determine an appropriate lane that is available to place the tumor specimen. In some embodiments, the process 1100 can execute at least a portion of 812 and/or 816 in the process 800 in FIG. 8.

At 1124, the process 1100 can determine which lane is available to place the specimen. In particular, for one or more flow cells each having the set of flow cell characteristics, the process 1100 can determine an appropriate lane that is available to place the non-tumor (e.g., normal) specimen. In some embodiments, the process 1100 can execute at least a portion of 812 and/or 816 in the process 800 in FIG. 8.

At 1128, the process 1100 can determine if a current pool is matched to the sample type of the specimen. Specifically, the process 1100 can populate one pool (e.g., the current pool) before moving on to populate another a liquid specimen pool. In some embodiments, the sample type can be a liquid or a solid. Furthermore, the process 1100 can place samples in an active pool because the process 1100 can run constantly and hold samples until the next applicable pool comes up. If the current pool is the same sample type as the specimen (e.g., the pool is a solid sample pool and the specimen is a solid sample), the process 1100 can proceed to 1132. If the current pool is not of the same sample type as the specimen (e.g., the pool is a solid sample pool and the specimen is a liquid sample), the process 1100 can proceed to 1136.

At 1132, the process 110 can place the specimen at an appropriate position in the current pool. In some embodiments, the process 1100 can execute at least a portion of 820 of the process 800 in FIG. 8. In some embodiments, the process 1100 can output the current pool. In some embodiments, the process 1100 can output the current pool to a flow cell allocation system. The flow cell allocation system can be configured to arrange the specimen in the flow cell and/or sequence the flow cell. In some embodiments, the process 1100 can output the at least one pool to a display for viewing by a lab technician. In this way, the lab technician can view a graphical representation of the at least one pool. In some embodiments, the process 1100 can arrange the at least one sample in the flow cell (e.g., if the process 1100 is executed in an automated sequencing system). In some embodiments, the process 1100 can sequence the flow cell to generate sequencing information for the specimen. In some embodiments, the process 1100 can display where specimens can be found to the lab technician.

At 1136, the process 1100 can wait until the current pool matches the sample type of the specimen. In some embodiments, once the current pool matches the sample type of the specimen, the process 1100 can proceed to 1132.

At 1140, the process 1100 can select a flow cell associated with the second assay configuration. In some embodiments, the second assay configuration can be associated with a blood sample type. In some embodiments, the process 1100 can receive flow cell data. The flow cell data can include a set of flow cell characteristics associated with a flow cell. In some embodiments, the flow cell characteristics can include a maximum capacity, a minimum capacity, and/or a number of lanes. In some embodiments, the flow cell can be an Illumina flow cell.

At 1144, the process 1100 can determine which lane is available to place the specimen. In particular, for one or more flow cells each having the set of flow cell characteristics, the process 1100 can determine an appropriate lane that is available to place the specimen. In some embodiments, the process 1100 can execute at least a portion of 812 and/or 816 in the process 800 in FIG. 8.

At 1148, the process 1100 can place the specimen at an appropriate position in a current pool. In some embodiments, the process 1100 can execute at least a portion of 820 of the process 800 in FIG. 8. In some embodiments, the process 1100 can output the current pool. In some embodiments, the process 1100 can output the current pool to a flow cell allocation system. The flow cell allocation system can be configured to arrange the specimen in the flow cell and/or sequence the flow cell. In some embodiments, the process 1100 can output the at least one pool to a display for viewing by a lab technician. In this way, the lab technician can view a graphical representation of the at least one pool. In some embodiments, the process 1100 can arrange the at least one sample in the flow cell (e.g., if the process 1100 is executed in an automated sequencing system). In some embodiments, the process 1100 can sequence the flow cell to generate sequencing information for the specimen. In some embodiments, the process 1100 can display where specimens can be found to the lab technician.

At 1152, the process 1100 can select a flow cell associated with the Xth assay configuration. In some embodiments, the process 1100 can receive flow cell data. The flow cell data can include a set of flow cell characteristics associated with a flow cell. In some embodiments, the flow cell characteristics can include a maximum capacity, a minimum capacity, and/or a number of lanes. In some embodiments, the flow cell can be an Illumina flow cell.

At 1156, the process 1100 can arrange the specimen in a pool in the flow cell. In some embodiments, the process can execute at least a portion of 1116-1136. It is understood that 1152 and 1156 are optional and may not be included in at least some embodiments.

### EXAMPLES

### Example 1

In this example, twelve samples are listed in Table 5 below, along with their masses and barcodes.

TABLE 5

| Sample Number | Mass (ng) | Barcode |
|---|---|---|
| S1 | 150 | AAB312 |
| S2 | 150 | AAB313 |
| S3 | 75 | AAB314 |
| S4 | 75 | AAB315 |
| S5 | 175 | AAB321 |
| S6 | 200 | AAB322 |
| S7 | 850 | AAB323 |
| S8 | 875 | AAB344 |
| S9 | 825 | AAB346 |
| S10 | 625 | AAB347 |
| S11 | 775 | AAB311 |
| S12 | 950 | AAB311 |

Each sample has an assigned barcode: S1-S10 have unique barcodes, and S11 and S12 have equal barcodes. Applying one of the heuristics discussed herein, S11 cannot share a lane with S12 because of their shared barcode.

In this example, the samples can be placed into one of three different flow cells that have different potential sizes measured in terms of minimum sample numbers, maximum sample numbers, and number of lanes. In particular, there is a first flow cell having one lane with twenty maximum samples and a required minimum of sixteen samples, a second flow cell having one lane with sixteen maximum samples and a required minimum of twelve samples, and a third flow cell having two lanes with sixteen maximum samples and a required minimum of twelve samples.

At 812, the process 800 determines that the first flow cell is invalid due to its minimum capacity of sixteen samples exceeding the total number of samples being evaluated. The process also determines that the second flow cell is invalid because, even though its required minimum of twelve samples is met by the number being evaluated, there are not enough lanes for the barcode clash between S11 and S12. Thus, the process determines that the third flow cell is the only valid one for evaluating the current list of samples.

At 816 and/or 820, the process can determine flow cell configurations for the samples, e.g., one or more pools for the third flow cell. In order to determine these configurations, the process may apply one or more of the heuristics discussed above to arrange the samples. For example, the process 800 can generate pools based on a mass threshold constraint. In this case, the threshold is set to 500 ng, so that the process 800 generates preliminary pools for S1-6 as a less than 500 ng group, and S7-12 as a greater than 500 ng group.

The process 800 can then generate permutations of potential lane allocations from each pool.

In doing so, the process may apply one or more of the heuristics described above to reduce the total number of permutations to be generated. For example, in order to avoid barcode clashing, the process may avoid generating any permutation that places samples S11 and S12 within the same lane. Similarly, while the third flow cell has sufficient capacity to analyze all twelve samples at once, in a situation in which the number of samples exceeds the maximum capacity of valid flow cells, the process may exclude permutations that do not include one or more high priority samples or may generate all permutations with high priority samples first and only then generate permutations using only lower-priority samples. In this case, exemplary permutations may include three “high mass” samples and three “low mass” samples per lane, five samples from one group (e.g., high mass) and one from another group (e.g., low mass) in each lane, or four samples from one group and two from another group. The process, however, will avoid generating permutations that all six samples from one group in a lane, since each of those permutations would include S11 and S12 together in a lane, causing a barcode clash.

From these permutations, the process 800 then can determine which permutation or set of permutations is preferred based on one or more criteria. Here, the process may determine that the permutations of the first option with three “high mass” samples and three “low mass” samples are better because they result in the least variation in pool size.

Using this subset of permutations, the process 800 can allocate samples as at step 824, e.g., according to the permutation that satisfies one or more other criteria. Those criteria may include, e.g., an acceptable or best mass balancing as among all of the pools in a lane, an acceptable or best mass balancing within each of the pools in a lane, an acceptable or best mass balancing as among pools of the same group in multiple lanes, an acceptable or best mass balancing as among multiple lanes, an arrangement that causes high priority samples to be evaluated more quickly or accurately, etc.

In this example, the process may select to allocate samples according to a preferred mass balancing within each pool in a lane. Using the allocation of three samples per group in a lane discussed above yields multiple possible permutations per lane. For example, a first such permutation may group (S1, S3, S6) as the low mass group in a first lane and (S2, S4, S5) as the low mass group in a second lane. Similarly, a second such permutation may group (S1, S5, S6) in one lane and (S2, S3, S4) in the second lane. However, applying the mass balancing within each pool heuristic, it will be seen that the second of these permutations is preferred as compared to the first:

TABLE 6

| | | Delta Between Min-Max |
|---|---|---|
| Permutation | Masses (ng) | Weight |
| S1, S3, S6 | 150, 75, 175 | 100 ng (175-75) |
| S2, S4, S5 | 150, 75, 200 | 125 ng (200-75) |
| S1, S5, S6 | 150, 175, 200 | 50 ng (200-150) |
| S2, S3, S4 | 150, 75, 75 | 75 ng (150-75) |

Thus, the method may include designating the first grouping as sub-optimal or non-optimal and the second grouping as optimal. Additionally or alternatively, the method also may include allocating 824 the samples of the low mass groupings into pools in separate lanes of the third flow cell according to the second permutation listed in Table 6.

### Example 2

In this example, the samples and steps up to and including the permutations generation are the same as in the previous example. Here, however, the system includes a cap on a maximum weight delta within a pool of 50 ng. In that situation, the first two permutations in Table 6 remain unacceptable because their deltas (100 ng and 125 ng) exceed that threshold. Now, the fourth permutation becomes unacceptable because its delta (75 ng) also exceeds that threshold.

In this situation, the system may determine that there is no allocation that includes two three low-mass sample pools that satisfy this criterion. In that case, the system may eliminate one or more of the criteria to move from finding an “optimal” solution to finding an acceptable one. In this case, the criterion to be removed may be the desire for an equal number of samples in each pool, so that the system may divide the last pool into separate pools, e.g., (S2) and (S3, S4), to yield three total pools (including (S1, S5, S6)).

### Example 3

Continuing from Example 2, the system may include further constraints, e.g., minimum or maximum pool sizes or masses. For example, the system may set a minimum pool size of two, which would cause S2 to be an invalid pool. (Doing so also would reduce the number of possible permutations to be calculated at the outset, as it would eliminate all pools of sample size one.) Thus, the acceptable solution may require reallocating samples instead of simply dividing one of the pools. In particular, the system may determine that the following allocations satisfy the “low weight” and “maximum delta within a pool of 50 ng or less” criteria:

TABLE 7

| | | Delta Between |
|---|---|---|
| Permutation | Masses (ng) | Min-Max Weight |
| S1, S2, S5, S6 | 150, 150, 200, 175 | 50 ng (200-150) |
| S3, S4 | 75, 75 | 0 ng (75-75) |

The system then may carry out a similar analysis for the high weight pools for Samples 7-12.

Each of the examples discussed above may be modified to account for other variable and/or heuristics built around those variables. Such variable may include, e.g., tissue source, fragmentation of the genetic material, processing conditions applied to the sample, priority of the sample, TAT, other processing costs, etc. Additionally or alternatively, the system may account for sample type, e.g., tumors, normals, and/or controls, and the constraints and/or pooling characteristics discussed above may be retained or modified depending on these variable and/or sample types.

The present disclosure has described one or more preferred embodiments, and it should be appreciated that many equivalents, alternatives, variations, and modifications, aside from those expressly stated, are possible and within the scope of the invention.

1. A method implemented on one or more computers having one or more processors for pooling a plurality of biological specimens for short read next generation sequencing, each specimen included in the plurality of specimens associated with a first plurality of specimen characteristics and a second plurality of specimen characteristics, the method comprising:
identifying a plurality of flow cell characteristics for each flow cell included in a plurality of flow cells;
selecting, by the one or more processors, one or more of the flow cells in the plurality of flow cells based at least in part on the plurality of flow cell characteristics and the first plurality of specimen characteristics, wherein the first plurality of specimen characteristics includes a mass of each specimen and at least one other specimen characteristic;
selecting, by the one or more processors, which specimens to put in each lane of each selected one or more of the flow cells based at least in part on the second plurality of specimen characteristics and at least one lane characteristic;
generating, by the one or more processors and for each lane including one or more specimens, at least one pool of specimens based at least in part on a placement of the selected specimens into respective lanes of the selected one or more of the flow cells; and
outputting, by the one or more processors, the generated pools.

2. (canceled)

3. The method of claim 1, wherein the outputting the generated pools comprises:
outputting the generated pools to a flow cell allocation system.

4. The method of claim 1, wherein the outputting the generated pools comprises:
outputting the generated pools to a display for viewing by a lab technician.

5. (canceled)

6. The method of claim 1 further comprising:
sequencing the at least one specimen in each outputted pool to generate sequencing information.

7. The method of claim 1, wherein a flow cell characteristic in the plurality of flow cell characteristics is an availability of a flow cell in the plurality of flow cells, wherein when a flow cell is unavailable, the method further comprises:
delaying sequencing the unavailable flow cell until the unavailable flow cell becomes available.

8. The method of claim 1, wherein the plurality of specimens comprises blood sample specimens, and the at least one pool in a selected lane comprises at least a portion of the blood specimens.

9. The method of claim 1, wherein the plurality of specimens comprises liquid sample specimens, and the at least one pool in a selected lane comprises at least a portion of the liquid specimens.

10. The method of claim 9, wherein the selected lane including the pool comprising at least a portion of the liquid specimens has a second pool comprising solid samples.

11. The method of claim 1, wherein the outputting the generated pools comprises:
outputting the generated pools to a whole genome processing system.

12. (canceled)

13. (canceled)

14. The method of claim 1, wherein
the step of selecting one or more of the flow cells includes evaluating a number of specimens included in the plurality of specimens in addition to evaluating the plurality of flow cell characteristics and the first plurality of specimen characteristics,
and wherein the step of selecting which specimen to put in each lane of each selected one or more of the flow cells includes:
determining a first plurality of flow cell specimen configurations based on the second plurality of specimen characteristics and the at least one lane characteristic, each of the first plurality of flow cell specimen configurations configured to house each specimen included in the plurality of specimens; and
determining a second plurality of flow cell specimen configurations based on the second plurality of specimen characteristics, the at least one lane characteristic, and at least one constraint, each flow cell specimen configuration included in the second plurality of flow cell specimen configurations comprising a flow cell specimen configuration included in the first plurality of flow cell specimen configurations.

15. The method of claim 14, wherein the plurality of flow cell characteristics comprises a turnaround time, the step of selecting which specimens to put in each lane of each selected one or more of the flow cells comprises:
generating, a plurality of hold costs based on the turnaround time associated with each specimen included in the plurality of specimens, each specimen included in the plurality of specimens being associated with a hold cost included in the plurality of hold costs, and the second plurality of flow cell specimen configurations being determined based on the plurality of hold costs; and
allocating specimens included in a specific flow cell specimen configuration included in the second plurality of flow cell specimen configurations to the at least one flow cell associated with the specific flow cell specimen configuration.

16. The method of claim 14, wherein the first plurality of specimen characteristics further comprise a unique identifier, a source tissue type, an age, and a turn-around-time.

17. The method of claim 14, wherein the plurality of flow cell characteristics comprises a maximum capacity, a minimum capacity, and a number of lanes, the first plurality of specimen characteristics comprises a barcode, and the step of selecting one or more of the flow cells comprises:
generating a preliminary set of flow cell groups comprising unique combinations of one or more flow cells included in the plurality of flow cells;
determining a secondary set of flow cell groups based on a number of specimens included in the plurality of specimens comprising at least a portion of the preliminary set of flow cell groups by, for each flow cell group included in the secondary set of flow cell groups:
determining that the maximum capacity of each flow cell included in the flow cell group is not exceeded by the number of specimens; and
determining that the minimum capacity of each flow cell included in the flow cell group is satisfied by the number of specimens;
determining a maximum number of repeated barcodes based on the barcode associated with each specimen in the plurality of specimens; and
determining a tertiary set of flow cell groups by determining, for each flow cell group included in the secondary set of flow cell groups, that a sum of the number of lanes associated with each flow cell in the flow cell group is at least as great as the maximum number of repeated barcodes, the set of flow cell groups comprising the tertiary set of flow cell groups.

18. The method of claim 14, wherein the plurality of flow cell characteristics comprises a maximum capacity, a minimum capacity, and a number of lanes, the second plurality of specimen characteristics comprises a barcode, and the determining the first plurality of flow cell specimen configurations comprises:
determining, for a specific flow cell specimen configuration included in the first plurality of flow cell specimen configurations, that the maximum capacity and the minimum capacity of each flow cell included in the specific flow cell specimen configuration can be satisfied while keeping specimens associated with equal barcodes in different lanes included in the specific flow cell specimen configuration based on the number of lanes associated with each flow cell in the target flow cell specimen configuration and the barcode associated with each specimen included in the plurality of specimens.

19. The method of claim 14, wherein the second plurality of specimen characteristics comprises the specimen type, wherein the specimen type is selected from a tumor, a normal match, and a control, and the determining the first plurality of flow cell specimen configurations comprises:
determining, for each flow cell specimen configuration included in the first plurality of flow cell specimen configurations, a processing cost based on the specimen type associated with the flow cell specimen configuration and the flow cell data; and
ranking each flow cell specimen configuration included in the first plurality of flow cell specimen configurations based on the processing cost associated with the flow cell specimen configuration.

20. The method of claim 14, wherein the plurality of flow cell characteristics comprises a turnaround time and the determining the second plurality of flow cell specimen configurations comprises:
generating, from the second plurality of flow cell specimens, unique pairs of specimens;
generating a plurality of compatibility scores based on the specimen data, each unique pair of specimens being associated with a compatibility score included in the plurality of compatibility scores;
generating a plurality of hold costs based on the turnaround time associated with each specimen included in the plurality of specimens, each hold cost included in the plurality of hold costs being associated with a specimen included in the plurality of specimens; and
wherein the step of generating at least one pool of specimens comprises generating a plurality of pools based on the plurality of compatibility scores and the plurality of hold costs, each pool included in the plurality of pools comprising at least one specimen.

21. The method of claim 20, wherein the step of selecting one or more of the flow cells further comprises:
generating a plurality of preliminary flow cell specimen configurations based on at least a portion of the plurality of pools and the at least one constraint, the second plurality of flow cell specimen configurations comprising at least a portion of the plurality of preliminary flow cell specimen configurations.

22. The method of claim 14, wherein the step of generating at least one pool of specimens comprises:
generating a first set of pools based on the second plurality of specimen characteristics, the at least one lane characteristic, and a first constraint included in the at least one constraint, each pool included in the first set of pools comprising at least one specimen included in the plurality of specimens; and
generating a second set of pools comprising a subset of the first set of pools based on the second plurality of specimen characteristics, the at least one lane characteristic, and a second constraint included in the at least one constraint.

23. The method of claim 22, wherein the step of generating at least one pool of specimens further comprises:
determining a group of one or more pools included in the second set of pools that satisfies the second constraint, and wherein the allocating the at least a portion of the specimens included in the plurality of specimens to one or more flow cells included in the plurality of flow cells comprises:
placing each pool included in the group of one or more pools into one or more lanes included in the one or more flow cells.

24. The method of claim 14, wherein the at least one constraint comprises at least one of a maximum pool mass, a maximum lane mass, a maximum flow cell mass, a minimum pool mass, a minimum lane mass, a minimum flow cell mass, a mass balancing threshold, a turnaround time constraint, a prioritization level, or a flow cell cost.

25. The method of claim 14, wherein the at least one constraint comprises a hard constraint.

26. The method of claim 14, wherein the at least one constraint comprises a soft constraint.

27. A biological specimen pooling system for pooling a plurality of biological specimens for short read next generation sequencing, each specimen included in the plurality of specimens associated with a first plurality of specimen characteristics and a second plurality of specimen characteristics, the system comprising at least one processor and at least one memory comprising instructions to:
identify a plurality of flow cell characteristics for each flow cell included in a plurality of flow cells;
select, by the one or more processors, one or more of the flow cells in the plurality of flow cells based at least in part on the plurality of flow cell characteristics and the first plurality of specimen characteristics, wherein the first plurality of specimen characteristics includes a mass of each specimen and at least one other specimen characteristic;
select, by the one or more processors, which specimens to put in each lane of each selected one or more of the flow cells based at least in part on the second plurality of specimen characteristics and at least one lane characteristic;
generate, by the one or more processors and for each lane including one or more specimens, at least one pool of specimens based at least in part on a placement of the selected specimens into respective lanes of the selected one or more of the flow cells; and
output, by the one or more processors, the generated pools.

28. The system of claim 27, wherein the memory further comprises instructions to:
as part of the step of selecting one or more of the flow cells, evaluate a number of specimens included in plurality of specimens in addition to evaluating the plurality of flow cell characteristics and the first plurality of specimen characteristics,
and, as part of the step of selecting which specimens to put in each lane of each selected one or more of the flow cells, determine a first plurality of flow cell specimen configurations based on the second plurality of specimen characteristics and the at least one lane characteristic, each of the first plurality of flow cell specimen configurations configured to house each specimen included in the plurality of specimens; and
determine a second plurality of flow cell specimen configurations based on the second plurality of specimen characteristics, the at least one lane characteristic, and at least one constraint, each flow cell specimen configuration included in the second plurality of flow cell specimen configurations comprising a flow cell specimen configuration included in the first plurality of flow cell specimen configurations.

29. The system of claim 27, wherein the memory further comprises instructions to:
output the at least one pool to a flow cell allocation system.

30. A non-transitory computer-readable storage medium having stored thereon program code instructions that, when executed by a processor, cause the processor to perform a method for pooling a plurality of biological specimens for short read next generation sequencing, each specimen included in the plurality of specimens associated with a first plurality of specimen characteristics and a second plurality of specimen characteristics, the method comprising:
identifying a plurality of flow cell characteristics for each flow cell included in a plurality of flow cells;
selecting, by the one or more processors, one or more flow cells in the plurality of flow cells based at least in part on the plurality of flow cell characteristics and the first plurality of specimen characteristics, wherein the first plurality of specimen characteristics includes a mass of each specimen and at least one other specimen characteristic;
selecting, by the one or more processors, which specimens to put in each lane of each selected one or more of the flow cells based at least in part on the second plurality of specimen characteristics and at least one lane characteristic;
generating, by the one or more processors and for each lane including one or more specimens, at least one pool of specimens based at least in part on a placement of the selected specimens into respective lanes of the selected one or more of the flow cells; and
outputting, by the one or more processors, the generated pools.

31. The method of claim 3, further comprising:
arranging, by the flow cell allocation system, the plurality of specimens in associated pools.

32. The method of claim 4, further comprising:
arranging, by the lab technician, the plurality of specimens in associated pools.
